(19) United States
(12) Patent Application Publication
(10) Pub. No.: US 2024/0225465 A1
(43) Pub. Date: Jul. 11, 2024

(54) PATIENT-WEARABLE DEVICE FOR DETECTING A SUBPULSE OF A PATIENT AND RELATED SYSTEMS, METHODS AND COMPUTER PROGRAM PRODUCTS

(71) Applicant: Dandelion Medical Devices, Inc., Vero Beach, FL (US)

(72) Inventors: Paul ADAMS, Vero Beach, FL (US); Christopher VETTER, Dublin, OH (US); Alexander PERAIRE-BUENO, Bedford, MA (US); Michael Andrew HOLTMAN, McLean, VA (US)

(73) Assignee: Dandelion Medical Devices, Inc., Vero Beach, FL (US)

(21) Appl. No.: 18/617,157

(22) Filed: Mar. 26, 2024

Related U.S. Application Data
(63) Continuation-in-part of application No. 18/055,052, filed on Nov. 14, 2022.

(51) Int. Cl. A61B 5/024; A61B 5/00

(52) U.S. Cl. CPC A61B 5/02438; A61B 5/0002; A61B 5/6833; A61B 2562/0204; A61B 2562/0219; A61B 2562/04; A61B 2562/06

(57) ABSTRACT

A patient-wearable device for detecting a subpulse of a patient and determining a pulse condition based thereon, and related systems, methods and computer program products. The device includes a base layer comprising a printed circuit board (PCB) and electronics connected thereto, and an adhesive layer connected to the base layer. The electronics may include one or more sensors that generate sensor data, a computer that processes the sensor data to determine the pulse condition of the patient, and a user interface (UI) component that generates a user-perceptible indication of the determined pulse condition. In alternate embodiments, the computer and the UI component may be external to the device and the device may communicate the sensor data to the computer via wired or wireless connection. In further embodiments, multiple devices may be attached to the patient and concurrently transmit raw or processed sensor data to facilitate determination of the pulse condition.

### CROSS-REFERENCE TO RELATED APPLICATIONS

This application is a continuation-in-part of U.S. patent application Ser. No. 18/055,052, filed Nov. 14, 2022, the entirety of which is incorporated by reference herein.

### BACKGROUND

Manual palpation of a pulse, also referred to as a pulse check, is the hallmark of cardiopulmonary resuscitation. Despite its simplicity, few people can accurately determine whether a patient is pulseless within an appropriately short period of time. Studies show that medical practitioners' success rates in rapidly performing a carotid pulse check on a pulseless patient is only in the upper teens (17%), while overall trained medical professionals generally are 55% accurate in manually palpating the presence of a pulse. Further, pulse palpation on individuals on extracorporeal devices has been shown to be only around 78% accurate with a mean time to decision at just over 20 seconds. It has also been reported that only 2% of first responders are able to recognize a truly pulseless patient within 10 seconds of evaluation, while 45% of first responders took 30 seconds to incorrectly determine a patient to be pulseless.

Because the medical mantra “time is tissue” pushes the medical community to minimize time to diagnosis, inaccurate and lengthy pulse detection presents a dilemma for cardiac resuscitation. The hallmark of a common cardiac rhythm during cardiopulmonary resuscitation, pulseless electrical activity (PEA), is in fact reliant on the detection of a pulse while still visualizing non-perfusing cardiac rhythm on a cardiac monitor. Discordance between a failure to palpate a pulse and the presence of a pulse leads to incorrect treatment management, prolongation of rhythm checks, or even abandonment of resuscitative efforts leading to patient death.

The most common locations for pulse palpation in a critically ill patient are the carotid arteries in the neck and the femoral arteries in the groin. Advanced Trauma Life Support (ATLS) guidelines support that a carotid pulse is palpable at a systolic blood pressure (SBP) of 60-70 mmHg and a femoral pulse at a SBP of 70-80. There are instances, however, where SBP is less than a reliably palpable level and as low as 42 mmHg and 52 mmHg, respectively. Critically, this discrepancy may cause providers to stop resuscitation and pronounce a patient dead with no palpable pulse even though the patient may simply have SBP less than 60 mmHg, and has a blood pressure that is perfusing organs. This scenario exemplifies the clinical “subpulse”—i.e., a spectrum of pulse that is less than reliably manually palpable. Such a patient with cardiac activity and a subpulse needs immediate vasopressor support and additional resuscitation, and not the standard resumption of compressions or cessation of resuscitation, both of which can cause harm. Apart from low SBP, accuracy of pulse and subpulse palpation is further dramatically affected by body habitus, provider experience, environmental stress, and strength of pulse which is directly related to blood pressure but also preexisting vascular disease.

While manual palpation of a pulse remains the guideline standard, recent advancements with use of doppler ultrasound have encouraged some practitioners to use such devices to determine the presence of a pulse. This has been shown to increase pulse detection accuracy to higher levels. Doppler ultrasound usage, however, presents two key problems. First, it requires an appropriate ultrasound unit to be on hand when a pulse check situation arises, and second, use of the ultrasound requires a dedicated practitioner, which keeps that practitioner from other resuscitation activities. Use of optical sensors in pulse oximeters is another recent development with the capability to monitor a host patient blood data, including pulse. Multiparameter patient monitor systems employing optical sensors, which typically display the pulse rate, are insufficient alone for pulse checks or in situations with decreased vascular flow. In particular, optical sensors are not adequate for detecting the subpulse. Optical sensors for medical utilization function during optimal conditions, such as minimal subcutaneous tissue between sensor and vessel (radial artery, fingertips, nasal, earlobe), and consistent strength of arterial pulse. Optical sensors are suboptimal/fail with decreased pulse strength and non-perfusion rhythms within the range of subpulse. Patient variability in blood pressure (strength of pulse), body mass, peripheral vascular disease, skin pigmentation and accessible vascular access limit the reliability of optical sensors and, critically, the unreliability or failure of optical sensors to detect subpulse.

Additionally, the determination of a strength and/or presence of a pulse is a common and vitally important examination practice in patients with peripheral vascular disease, which inflicts over 8 million people in the United States and 200 million globally and is the manifestation of systemic atherosclerosis that progressively occludes arteries with atherosclerotic plaque. A common and important practice is palpation of peripheral pulses during each doctor's evaluation. A decreased or absent pulse from the baseline pulse can be a medical emergency and represent near or total vascular occlusion. Typically, a practitioner will initially attempt to palpate a pulse, however the nature of vascular disease significantly decreases the blood flow to the distal artery, leading to decreased pulse strength and difficulty with manual pulse palpation. A provider may inaccurately reason the pulse is absent, however a subpulse may in fact be present. Current standard of care involves using a Doppler ultrasound machine to methodically locate a subpulse. This can be time and labor intensive, and have significant provider variability, as small Doppler surface area requires precise knowledge of arterial location. Further, the force applied with the Doppler can occlude the pulse that leads to inaccurately concluding the absence of a pulse, and the low strength of a subpulse is reliant on the provider hearing the acoustic signal of the Doppler, which is further limited by loud and chaotic environments.

Overall, the current standards for pulse detection and subpulse detection in particular are inaccurate, subjective, and burdensome, the results of which can lead to inappropriate medical decisions and patient harm, especially with critically ill patients.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

A patient-wearable device is described herein for detecting a subpulse of a patient and determining a pulse condition based thereon, as well as related systems, methods and computer program products. In an embodiment, the patient-wearable device includes a base layer comprising a printed circuit board (PCB) and electronics connected thereto, and an adhesive layer that is connected to the base layer, the adhesive layer comprising an adhesive suitable for attaching the patient-wearable device to a location on a body of the patient. The electronics may include one or more sensors that generate sensor data, a computer that is connected to the one or more sensors and processes the sensor data generated thereby to determine the pulse condition of the patient, and a user interface (UI) component that is connected to the computer and controlled thereby to generate a user-perceptible indication of the determined pulse condition. In alternate embodiments, the computer and the UI component may be external to the patient-wearable device and the patient-wearable device may communicate the sensor data to the computer via a wired or wireless connection. In further embodiments, multiple patient-wearable devices may be attached to the patient and concurrently transmit raw or processed sensor data to determine the pulse condition.

Further features and advantages of the embodiments, as well as the structure and operation of various embodiments, are described in detail below with reference to the accompanying drawings. It is noted that the claimed subject matter is not limited to the specific embodiments described herein. Such embodiments are presented herein for illustrative purposes only. Additional embodiments will be apparent to persons skilled in the relevant art(s) based on the teachings contained herein.

### BRIEF DESCRIPTION OF THE DRAWINGS

The accompanying drawings, which are incorporated herein and form a part of the specification, illustrate embodiments of the present application and, together with the description, further serve to explain the principles of the embodiments and to enable a person skilled in the pertinent art to make and use the embodiments.

FIG. 1 illustrates a perspective view of a patient-wearable device for detecting a pulse condition of a patient in accordance with an embodiment.

FIG. 2 illustrates a side view of the device shown in FIG. 1.

FIG. 3 illustrates a perspective view of a patient-wearable device for detecting a pulse condition of a patient that includes at least one light emitting diode (LED) indicator in accordance with an embodiment.

FIG. 4 illustrates an exploded view of an electronic assembly of a patient-wearable device for detecting a pulse condition of a patient in accordance with an embodiment.

FIG. 5 depicts a flowchart of a method for detecting a pulse condition of a patient in accordance with an embodiment.

FIG. 6 illustrates a system for providing detection of a pulse condition of a patient in accordance with an embodiment.

FIG. 7 depicts a sheet of patient-wearable devices for detecting a pulse condition of a patient prior to application thereof in accordance with an embodiment.

FIG. 8 illustrates a side view of a patient-wearable device for detecting a pulse condition of a patient in accordance with an embodiment.

FIG. 9 depicts an exemplary implementation of a computing device that may be used to implement embodiments described herein.

FIG. 10 illustrates a side view of a patient-wearable device for detecting a pulse condition of a patient in accordance with an embodiment.

FIG. 11 illustrates a patient-wearable device for detecting a pulse condition of a patient in accordance with an embodiment.

FIG. 12 illustrates a patient-wearable device for detecting a pulse condition of a patient in accordance with an embodiment.

FIG. 13 illustrates a top and side perspective view of a patient-wearable device for detecting a pulse condition of a patient in accordance with an embodiment.

FIG. 14 illustrates a cross-sectional side view of a patient-wearable device for detecting a pulse condition of a patient in accordance with an embodiment.

FIG. 15 illustrates cross-sectional side view of a patient-wearable device for detecting a pulse condition of a patient in accordance with an embodiment.

FIG. 16 illustrates a cross-sectional side view of a patient-wearable device for detecting a pulse condition of a patient in accordance with an embodiment

FIG. 17 illustrates a cross-sectional side view of a patient-wearable device for detecting a pulse condition of a patient in accordance with an embodiment.

FIG. 18 illustrates a cross-sectional side view of a patient-wearable device for detecting a pulse condition of a patient in accordance with an embodiment.

FIG. 19 depicts a flowchart 1900 of a method for selectively activating/deactivating sensors of a plurality of patient-wearable devices that are attached or attachable to different locations on a body of a patient, in accordance with an embodiment.

FIGS. 20A, 20B, 20C and 20D illustrate different patient-wearable device configurations that may be suitable for attachment to respective different locations on a body of a patient, in accordance with embodiments.

The features and advantages of the embodiments described herein will become more apparent from the detailed description set forth below when taken in conjunction with the drawings, in which like reference characters identify corresponding elements throughout. In the drawings, like reference numbers generally indicate identical, functionally similar, and/or structurally similar elements. The drawing in which an element first appears is indicated by the leftmost digit(s) in the corresponding reference number.

### DETAILED DESCRIPTION OF THE EMBODIMENTS

### I. Introduction

The following detailed description discloses numerous example embodiments. The scope of the present patent application is not limited to the disclosed embodiments, but also encompasses combinations of the disclosed embodiments, as well as modifications to the disclosed embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future.

References in the specification to “one embodiment,” “an embodiment,” “an example embodiment,” “another embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

In the discussion, unless otherwise stated, adjectives such as “substantially” and “about” modifying a condition or relationship characteristic of a feature or features of an embodiment of the disclosure, are understood to mean that the condition or characteristic is defined to within tolerances that are acceptable for operation of the embodiment for an application for which it is intended.

Furthermore, it should be understood that spatial descriptions (e.g., “above,” “below,” “up,” “left,” “right,” “down,” “top,” “bottom,” “vertical,” “horizontal,” etc.) used herein are for purposes of illustration only, and that practical implementations of the structures and drawings described herein can be spatially arranged in any orientation or manner Additionally, the drawings may not be provided to scale, and orientations or organization of elements of the drawings may vary in embodiments.

The various embodiments set forth herein are described in terms of exemplary block diagrams and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

Numerous exemplary embodiments are described as follows. It is noted that any section/subsection headings provided herein are not intended to be limiting. Embodiments are described throughout this document, and any type of embodiment may be included under any section/subsection. Furthermore, embodiments disclosed in any section may be combined with any other embodiments described in the same section and/or a different section.

### II. Example Embodiments

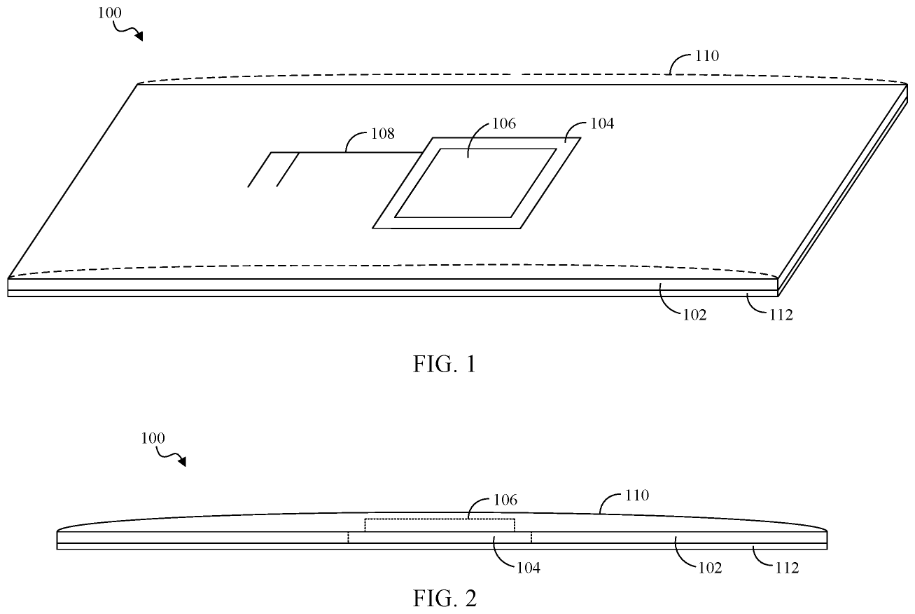

FIG. 1 illustrates a perspective view of a patient-wearable device 100 for detecting a pulse condition of a patient in accordance with an embodiment. As used herein, the term “pulse condition” is intended to at least encompass the presence or absence of a pulse, as well as any characteristics of a detected pulse (e.g., pulse strength) or any characteristics or conditions determinable based on a detected pulse or absence thereof (e.g., heart rate, or presence of an occlusion). FIG. 2 illustrates a side view of device 100.

Device 100 is capable of detecting pulses of various strengths but, importantly, is capable (both through choice of sensor(s) and through post-processing of sensor data, as will be described herein) of detecting subpulses. As used herein, the term “subpulse” refers to a spectrum of pulse that is less than reliably manually palpable. As discussed in the Background Section above, the failure to accurately detect a pulse or determine the absence of a pulse can lead to inappropriate medical decisions and patient harm, especially with critically ill patients.

Device 100 may be of any practicable size as deemed desirable or suitable for a particular application, though it will generally be desirable to have the smallest size useful. As shown in FIGS. 1 and 2, device 100 is rectangularly-shaped. However, embodiments of device 100 can be of any shape. For example, device 100 may be triangular, square, round, oval, or irregularly shaped in nature such as a star configuration or web configuration, which may be optimal for certain procedures. It may be that device 100 is associated with a larger surface area attachment than that shown in FIG. 1 for a desired pulse condition detection. A larger attached system may also contain multiple devices within it that perform the same or separate function as described herein in reference to device 100.

Device 100 comprises a flexible printed circuit board (PCB) 102. Flexible PCB 102 may be a two-layer PCB—however, this is an example only and flexible PCB 102 may comprise only a single layer or more than two layers. As shown in FIGS. 1 and 2, device 100 also includes electronics 106 that are mounted on or otherwise connected to PCB 102. As will be discussed herein, electronics 106 includes one or more sensors and may also include a microcontroller (e.g., for processing sensor data and/or transmitting unprocessed or processed sensor data to an external device or system), and a power source such as a battery. As further shown in FIG. 1, flexible PCB 102 comprises a stiffener 104 in a region thereof to which electronics 106 are connected. Stiffener 104 may provide improved stability and support for electronics 106. Stiffener 104 may be implemented using a material such as FR4, polyimide, aluminum or stainless steel, although these are only examples and are not intended to be limiting. Stiffener 104 may be attached to PCB 102 using thermal bonding, pressure sensitive adhesives, or any other suitable attachment method.

Device 100 further includes an antenna 108 formed on or connected to flexible PCB 102 for enabling unidirectional or bidirectional communication between device 100 (e.g., a microcontroller of device 100) and one or more external devices. Antenna 108 may comprise, for example, a trace antenna that is formed directly on the surface of flexible PCB 102 or a ceramic chip antenna that is mounted on PCB 102.

An overlay 110 to flexible PCB 102 may further be provided and can include any materials that will not interfere with the functioning of electronics 106 and antenna 108, and may protect electronics 106 and antenna 108. In one embodiment, overlay 110 is composed of silicone, although other materials may be used. The thickness of device 100 can vary and may be dictated by a size of a largest contained component, such as a battery, if included. However, it may be deemed desirable to maintain a thinnest thickness achievable for improved flexibility—accordingly, in some embodiments of device 100, such thickness can be in the millimeter(s) range.

In an alternate embodiment of device 100, flexible PCB 102 may be replaced by a flexible base sheet and a smaller semi-rigid PCB (single-layer or multi-layer) may be disposed (e.g., centrally) thereon or therein to support electronics 106. Such semi-rigid PCB may be square shaped, although other shapes may be used. Such semi-rigid PCB may be sufficiently small such that it can align with the contours and surface of a body part to which device 100 is applied. For example, such a semi-rigid PCB may be in the range of 5 to 50 mm square, and in certain embodiments may be in the size range of a 10 to 20 mm square. However, these are merely examples, and the semi-rigid PCB may be several hundred mm square, or other sizes suitable for an intended application. In an embodiment that includes the semi-rigid PCB, the flexible base sheet may support and/or surround the semi-rigid PCB. The material used in the flexible base sheet may be any suitable material for practicing embodiments described herein. The size of flexible PCB 102 or the flexible base sheet may be determined based on factors such as but not limited to increasing adhesion or achieving desired acoustical properties.

As further shown in FIG. 1, device 100 comprises an adhesive layer 112 that enables device 100 to be affixed to the body (e.g., the skin) of a patient. Adhesive layer 112 may comprise, for example and without limitation, a ready-for-use adhesive pad or film. The adhesive used for adhesive layer 112 can be any conventional adhesive appropriate for contact with a patient's skin. In certain embodiments, the adhesive used may also be conductive so as to enable or increase coupling between one or more sensors of device 100 and the patient. Although device 100 is shown as including adhesive layer 112 for affixing device 100 to a body of a patient, other alternative or additional means of maintaining contact between device 100 and a body of the patient can be used. For example, device 100 may be secured to the body using one or more of suction, cuffs, bands, ties, sprays, gravity, clips, etc., so long as interference with the sensors of device 100 is sufficiently low that a desired pulse condition detection function can be achieved.

In embodiments, adhesive layer 112 comprises a replaceable adhesive pad or film that may be attached to device 100 prior to application to a patient. The replaceable adhesive pad or film may be sterile. For example, the adhesive pad or film may be a pre-sterilized disposable component manufactured from relatively inexpensive materials. The pre-sterilized disposable component may be pre-packaged in a suitable packaging material that can be opened at time of use. In accordance with such an embodiment, when a use of device 100 with a particular patient is completed, the pre-sterilized disposable component may be discarded.

Although adhesive layer 112 is shown as being attached to the bottom of PCB 102 in FIG. 1, in alternate embodiments adhesive layer 112 may be disposed across the top of PCB 102 and extend off the sides thereof, or may surround PCB 102 and extend from the sides thereof, so long as adhesive layer 112 is connected (directly or indirectly) to PCB 102 and is enabled to come into contact with a patient's skin such that it can secure PCB 102 thereto.

The aforementioned base sheet and/or adhesive layer 112 of device 100 may be embedded with an additional matter to support device functioning. For instance, the base sheet may be impregnated with electrically conductive material, such as a flexible wire mesh or conductive adhesive, that aids in sensing. An embodiment may be adapted for ECG monitoring. Further, the presence of conductive material in the base sheet or adhesive layer 112 may further aid in communication of device 100 with other devices or external computers. In a still further example, device 100 may utilize the additional material within the base sheet or adhesive layer 112 as a mechanism by which a primary sensor functioning can be amplified. In this scenario, the added material may act to increase the surface area of a primary sensor and its contact points with the body of the patient. Materials in the substrate, or structures on device 100, may also be used to amplify the signal, such as in the case of vibration or sensing done with an accelerometer.

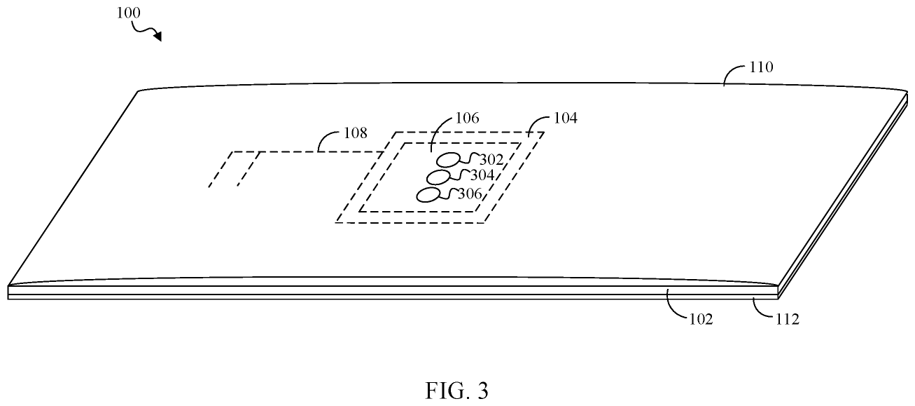

FIG. 3 illustrates a perspective view of an embodiment of device 100 that includes a number of light emitting diode (LED) indicators 302, 304 and 306 in accordance with an embodiment. Although the embodiment shown in FIG. 3 includes three LED indicators, it should be understood that device 100 may include any number of LED indicators as deemed necessary or desirable. Each LED indicator 302, 304 and 306 may be disposed on top of overlay 110 or may be partially or fully disposed in a cavity formed therein, so long as the LED indicator is visible to a practitioner. Furthermore, each LED indicator 302, 304 and 306 may be connected to flexible PCB 102 via a corresponding channel in overlay 110 such that the LED indicator can be powered on or off or otherwise controlled by other component(s) within electronics 106 (e.g., by a microcontroller within electronics 106).

Such LED indicator(s) may be used to for a variety of purposes, such as but not limited to visually indicating a pulse condition of the patient or signifying a status of device 100. A status of device 100 may include, for example, detecting a pulse, streaming (e.g., streaming sensor data to an external computer), powered on, powered off, sleeping (when device 100 supports a low-power sleep mode), functioning, malfunctioning, or the like. Different pulse conditions or statuses may be indicated by using different colors, illumination patterns, degrees of illumination, and/or numbers of LEDs activated.

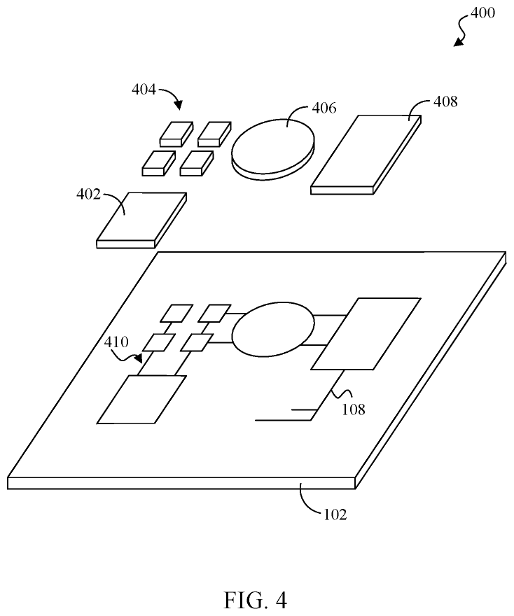

FIG. 4 illustrates an exploded view of an electronic assembly 400 that may be used to implement electronics 106 of device 100 in accordance with one example embodiment. As shown in FIG. 4, electronic assembly 400 includes a number of components that are connected to flexible PCB 102 (e.g., on stiffener 104 of flexible PCB 102) and also electronically connected to each other via a number of PCB traces formed on flexible PCB 102, collectively denoted PCB traces 410. These components include a sensor 402, a number of passive electronic components 404, a battery 406, a microcontroller 408, and antenna 108.

Sensor 402 may comprise any type of sensor suitable for detecting a pulse condition in a patient. In an embodiment, sensor 402 comprises an inertial measurement unit (IMU) that integrates one or more of a multi-axis accelerometer or multi-axis gyroscope and that provides suitable sensitivity to detect a desired pulse in a patient. In another embodiment, sensor 402 comprises an acoustic sensor. However, these are merely examples and other types of sensors may be used for detecting a desired pulse in a patient.

Although only a single sensor 402 is shown in FIG. 4 for the sake of illustration, it is to be understood that device 100 may contain any number of sensors that aid directly in pulse condition detection, or in the detection of other patient qualities or conditions. For example, device 100 may include a primary sensor of a first type (e.g., an IMU) and one or more additional sensors of a second type (e.g., acoustic sensors) that may be used to provide further sensing capabilities and/or to provide checks on the primary sensor. In certain settings, combined data, such as that from both inertial and acoustic sensing, may give enhanced data fidelity because information from each type of sensing is fundamentally different. In yet another embodiment, device 100 may comprise multiple sensors of a same type. For example, device 100 may comprise a plurality of physical accelerometers, each of which generates its own sensor data.

In embodiments, the sensors utilized by device 100 may comprise any one of the following sensor types having a sensitivity (alone or combined with other sensors) suitable for detecting a subpulse: an accelerometer, a gyroscope, a magnetometer, an IMU that comprises one or more of an accelerometer, a gyroscope or a magnetometer, or an acoustic sensor.

As noted above, device 100 may also include sensors for detecting patient qualities or conditions other than a pulse condition. For example, device 100 may include sensors for detection of one or more of blood pressure, blood sugar, blood oxygen (e.g., a pulse oximeter), echocardiogram, body temperature, respiratory rate, blood flow rate, magnetic fields, or the like.

Passive electronic components 404 comprise circuit components that do not require a power source (such as resistors, capacitors, inductors, and the like) and that are used to control the flow of power and electrical signals to the other electronic components that make up electronic assembly 400.

Battery 406 comprises a power source for active electronic components within electronic assembly 400. For example, battery 406 may be used to provide power for sensor 402 and microcontroller 408. In one embodiment, battery 406 comprises a button cell battery, although this is only one example.

Microcontroller 408 comprises an integrated circuit (IC) chip that implements a computer configured to perform various functions relating to detecting a pulse condition in a patient as will be described herein. In an embodiment, microcontroller 408 is wireless-enabled and thus may communicate wirelessly with one or more external devices (e.g., for the purpose of communicating sensor data and/or other information). For example, microcontroller 408 may be capable of communicating with other devices via a Bluetooth® protocol (e.g., as specified by the IEEE 802.15.1 standard), a Wi-Fi® protocol (e.g., as specified by the IEEE 802.11 family of standards), and/or other radio frequency (RF) protocol. Hospital settings may dictate a preferred form of wireless communication for device 100; however, Wi-Fi® is believed to be sufficiently robust in most clinical settings so as to not interfere with other patient devices or equipment.

In various embodiments, device 100 may include a microprocessor, a digital signal processor (DSP), or an application-specific integrated circuit (ASIC) instead of microcontroller 408, or in addition to microcontroller 408, for performing processing tasks.

As shown in FIG. 4, to facilitate the aforementioned wireless communication, electronic assembly 400 includes antenna 108 that is connected to microcontroller 408. In the embodiment shown in FIG. 4, antenna 108 comprises a trace antenna that is formed directly on flexible PCB 102 in a well-known manner However, this is an example only, and antenna 108 may comprise a ceramic chip antenna or other suitable type of antenna.

In an alternate embodiment, device 100 may be capable of communicating with an external device via a wired connection. For example, in an embodiment, device 100 does not include microcontroller 104 but instead communicates sensor data to an external computer via a wired connection thereto. Such external computer may comprise, for example, and without limitation a microcontroller (e.g., an Intel® 8051 microcontroller), a microcontroller board (e.g., an Arduino® microcontroller board), or a microprocessor-based mini-computer (e.g., a Raspberry Pi® microprocessor-based mini-computer). In an alternate embodiment, the communication of the sensor data to the external computer is carried out via a wireless connection. In still further embodiments, device 100 may include microcontroller 104 and also communicate with an external computer via a wired and/or wireless connection thereto.

Embodiments of device 100 can be applied in all clinical settings, including for use during cardiopulmonary resuscitation (CPR), during cardiac arrest (code), or the moments just prior to or after cardiac arrest (peri-code), on patients with or without forms of vascular disease that impact pulse detection, to detect the presence of a pulse in an extremity for cases of concern for arterial clot, or pulse/heart rate detection in persons, including fetal heart rate/pulse.

In an embodiment, device 100 is suitable for use on a patient for an extended period of time, such as the duration of a stay at a hospital. For example, device 100 may be adapted to have a relatively large internal battery power source, be wired to an external power source, and/or enter an energy-saving rest mode during periods of non-use for activation when a pulse check is required. As another example, adhesive layer 112 may comprise a material that provides for long-term adhesion. Such long-term adhesion could be valuable during hospitalization or for telemedicine to determine dynamic changes of a pulse in real time for immediate provider notification. The clot of an artery (such as a radial artery or femoral artery occlusion) is a true medical emergency and needs to be diagnosed immediately.

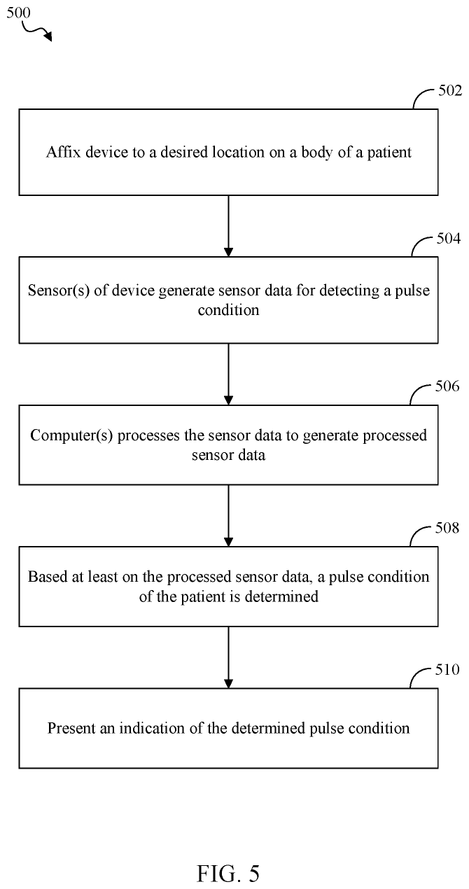

FIG. 5 depicts a flowchart 500 of a method for detecting a pulse condition of a patient in accordance with an exemplary embodiment. As shown in FIG. 5, the method of flowchart 500 begins at step 502 in which device 100 is affixed (e.g., by a practitioner) to a desired location on a body (e.g., on the skin) of a patient. As noted above, an adhesive layer 112 and/or various other means of attachment (e.g., suction, cuffs, bands, ties, sprays, gravity, clips) may be used to secure device 100 to a desired body location.

In embodiments, the size and flexibility of device 100 render it suitable for attachment to most locations on a body of a patient. In embodiments, device 100 may be suitable for attachment to any location on a patient's body, but in accordance with particular embodiments, device 100 can be attached at least over the superficial aspects of the dorsalis pedis (DP) artery (along the dorsal aspect of the foot) and the posterior tibial (PT) artery (posterior to the medial malleolus). By way of further example, device 100 may be placed along the popliteal artery (posterior to the knee in the popliteal fossa) and/or femoral artery (mid to medial aspect of the inguinal ligament (commonly the groin)). Device 100 may also be placed on the chest (possibly near the Point of Maximal Impulse (PMI)), over a carotid artery, over a femoral artery, or over a radial artery. The location of attachment of device 100 can yield different advantages. In some instances, the placement of device 100 may allow the determination of point of occlusion along the lower or upper extremity for example. Device 100 may be suitable for attachment to a body surface over or adjacent to an underlying vascular structure.

At step 504, after device 100 has been affixed to a location in step 502, one or more sensors of device 100 (e.g., sensor 402) generate sensor data for detecting a pulse condition. Such sensor(s) may include, but are not limited to, a multi-axis accelerometer, a multi-axis gyroscope, an IMU (e.g., that incorporates a multi-axis accelerometer and gyroscope), an acoustic sensor, a magnetometer, or any other type of sensor deemed suitable for detecting a pulse condition in a patient.

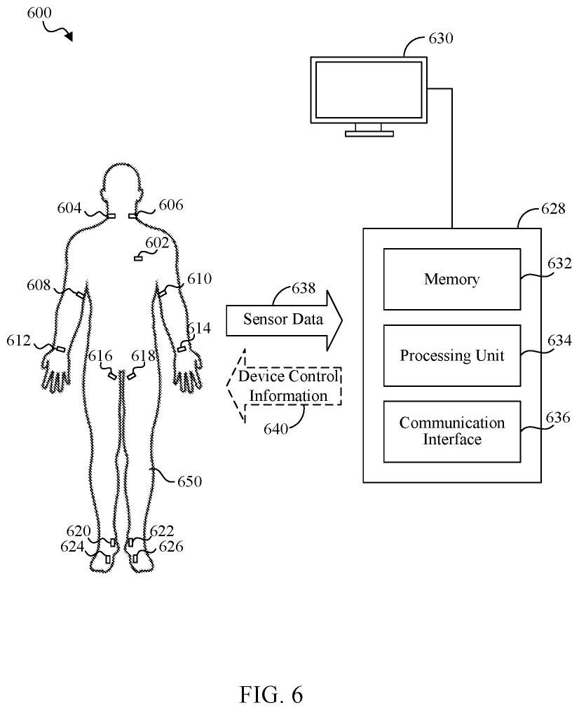

At step 506, the sensor data generated during step 504 is provided to one or more computer(s) and such computer(s) process the sensor data to generate processed sensor data. The computer(s) used to process the sensor data may be located on device 100 (e.g., in the form of microcontroller 408) or may be located externally with respect to device 100, in which case the sensor data generated during step 504 may be transmitted thereto via a wired or wireless connection. Still further, the processing of sensor data may be carried out in a distributed manner by a computer located on device 100 and one or more external computers. Various system implementations that rely on external computers for processing the sensor data will be described below in reference to FIG. 6.

The processing of the sensor data during step 504 may be carried out, for example, to address the issue of background noise, which can originate from a variety of sources in the clinical setting and can reduce overall accuracy of pulse readings. Such background noise may result from surface-level movements of the patient's body, both direct and indirect, as well active electronic monitoring, such as electrocardiograms (ECGs), cardiac monitors, pacemaker/defibrillator pads, and ultrasounds. Background noise can lead to significant rates of false positives where a perceived pulse detection is actually interference with the patient anatomy, such as simply lifting the patient's arm. Background noise can be addressed at least in part through the choice of sensor(s) that generate the sensor data in step 504. However, in embodiments, the issue of background noise is alternatively or additionally addressed through appropriate processing of the sensor data in step 506. For example, the computer(s) that process the sensor data may filter and/or compensate for background noise to reduce such false positive readings. In the case of filtering through sensor selection, complementary sensors that are vulnerable to noise in different domains can be used together to extract the target signal. Processing of the sensor data (e.g., analog or digital signal representations) may also be performed in either or both the time and frequency domains. Strategies may include, but are not limited to, pattern matching with expected heartbeat waveforms, filtering based on key heartbeat waveform attributes (duration, amplitude, etc.), and filtering of key frequencies in the frequency domain

Filtering of the sensor data in the time domain may include, for example and without limitation, removing noise that is far from an expected heartbeat. For example, in a scenario in which an accelerometer is used, such noise can be removed if there is a large spike in acceleration, which may be more likely due to movement (e.g., a cough) other than a heartbeat. An embodiment can also filter out the effects of movements that are unlike a heartbeat in terms of duration. For example, if there is a spike that lasts much longer than expected, it may be the patient breathing, rather than a heartbeat. The processing can be adjusted to greater and lesser extents depending on what is being looked for in terms of shape and amplitude of a target signal.

Filtering of the sensor data in the frequency domain may include, for example and without limitation, cleaning up a sensor-generated signal with band pass filters, by analyzing dominant frequencies in the signal, or the like. In some embodiments, a combination of filtering in the time domain and filtering in the frequency domain may be used to generate the processed sensor data.

Processing of the sensor data in step 506 may alternatively or additionally comprise comparing and/or combining sensor data generated by multiple different sensors of device 100 or comparing and/or combining sensor data generated by the sensor(s) of device 100 with sensor data provided from other devices that are attached to the patient. An example system that can process sensor data generated by multiple different devices that are simultaneously attached to a patient will be described below in reference to FIG. 6.

The aforementioned processing of the sensor data in step 504 can enhance the ability of device 100 or of a system including device 100 to detect a pulse condition in a patient, including a subpulse.

At step 508, a pulse condition of the patient is determined based at least on the processed sensor data. This step may be performed, for example, by any of the same computer(s) used to process the sensor data in step 506, or by a different computer that receives the processed sensor data therefrom. The determined pulse condition may include, for example and without limitation, a presence or absence of a pulse, a characteristic of a detected pulse (e.g., pulse strength), or a characteristic or condition determinable based on a detected pulse or absence thereof (e.g., heart rate, or presence of an occlusion).

At step 510, an indication of the determined pulse condition is provided (e.g., to a practitioner). For example, a visual and/or auditory indication of the determined pulse condition of the patient can be provided to a practitioner by one or more suitable user interface components of device 100, and/or by one or more suitable user interface components external to device 100.

For example, device 100 may include one or more LED indicators as discussed above in reference to FIG. 3 and such LED indicators may be used to visually indicate a determined pulse condition of a patient. For instance, illumination of a green LED indicator may indicate that a pulse has been detected while illumination of a red LED indicator may indicate that no pulse has been detected. Blinking vs. steady illumination of an LED indicator may also be used to distinguish between detection and non-detection of a pulse. A relatively strong pulse may be indicated by illuminating more LED indicators than would be illuminated for a relatively weak pulse. Likewise, a relatively strong pulse may be indicated by a brighter illumination of an LED indicator while a relatively weak pulse may be indicated by a dimmer illumination of the LED indicator. Furthermore, an LED indicator may be illuminated in a periodic manner that mimics a detected pulse (e.g., with the LED indicator lighting up for each beat of the pulse). However, these are only examples, and persons skilled in the relevant art(s) will appreciate that any of a variety of LED indicator features (e.g., color, illumination pattern, degree of illumination, and/or number of LEDs activated) can be used to indicate a determined pulse condition to a practitioner.

In another example embodiment, device 100 may include a mini- or micro-speaker that is capable of emitting an auditory indicator of a pulse condition. For example, the speaker may emit a sound only if a pulse has been detected. As another example, the speaker may emit a first sound to indicate that a pulse is detected and emit a second sound to indicate that a pulse has not been detected. As yet another example, the speaker may emit sounds that mimic a perceived pulse of the patient upon detection.

In a further example embodiment, a visual and/or auditory indication of the detected pulse condition may be displayed via a display screen and/or speaker associated with an external computer to which device 100 is communicatively coupled via a wireless or wired communication medium.

FIG. 6 depicts a system 600 for providing detection of a pulse condition of a patient in accordance with an embodiment. As shown in FIG. 6, system 600 includes a plurality of patient-wearable devices (i.e., patient-wearable devices 602, 604, 606, 608, 610, 612, 614, 616, 618, 620, 622, 624 and 626) that are concurrently attached to different locations on a body of a patient 650, a computing device 628, and a display device 630.

As further shown in FIG. 6, computing device 628 comprises memory 632, a processing unit 634, and a communication interface 636. Memory 632 may comprise, for example, one or more volatile memory devices (e.g., one or more RAM devices) and/or one or more non-volatile memory devices (e.g., one or more ROM devices, flash memory devices, magnetic storage devices, optical disks, or the like). Processing unit 634 may comprise one or more microprocessors, microcontrollers, DSPs, and/or ASICs. Processing unit 634 may be configured to execute software instructions stored in memory 632 to perform any of the operations attributed to computing device 628 as described herein (e.g., processing of sensor data to generate processed sensor data, the detection of a pulse condition based on sensor data, and/or the generation of a visual or auditory indication of the detected pulse condition). Communication interface 636 may comprise a wired communication interface (e.g., a USB interface) and/or a wireless communication interface (e.g., a Bluetooth®, WiFi® or other RF interface) suitable for receiving sensor data 638 from one, some or all of the wearable devices shown in FIG. 6, and for optionally transmitting device control information 640 to one, some or all of the wearable devices shown in FIG. 6. Display device 630 may comprise a part of computing device 628 (e.g., integrated into a same housing as computing device 628) or may be separate from computing device 628 but connected thereto via a suitable wired or wireless connection.

Each of the patient-wearable devices in FIG. 6 may be implemented in a like manner to device 100 as discussed above. Furthermore, each such device may be capable of transmitting unprocessed and/or processed sensor data 638 to computing device 628 via a wired or wireless connection. Computing device 628 may receive such sensor data 638, e.g., via communication interface 636, and utilize such sensor data 638 to detect a pulse condition in patient 650 (e.g., a pulse condition for each one of the patient-wearable devices). Computing device 628 may then provide an indication of each one of the detected pulse conditions to a practitioner. For example, computing device 628 may cause a visual indication of each one of the detected pulse conditions to be displayed (e.g., concurrently) by display device 630.

The patient-wearable devices may thus be considered additive in nature and can be placed as desired throughout the anatomy of a patient (e.g., patient 650) to perform a specific sensing task. The ability to concurrently detect pulse conditions at different body locations may be particularly beneficial in situations of cardiac arrest and other critical conditions for which rapid pulse detection (or lack thereof) is pivotal. For example, different patient-wearable devices may be concurrently attached to the chest (possibly near the Point of Maximal Impulse (PMI)), over a carotid artery, over a femoral artery, and/or over a radial artery. Several points of contact may increase sensitivity and specificity and allow for additional clinical decisions based on data points and calculations. For example, an aortic dissection may be indicated if pulse strength readings from patient-wearable devices placed on the left side of body are different than pulse strength readings from patient-wearable devices placed on the right side of the body. Multiple points of body contact may increase accuracy and also allow real time data to be collected for oxygenation levels, body temperature, respiratory rate, and change in blood pressure.

In an embodiment, each of the patient-wearable devices shown in FIG. 6 transmits unprocessed sensor data to computing device 628 via communication interface 636, and processing unit 634 of computing device 628 processes and interprets the sensor data to detect a pulse condition. Processing unit 634 then generates a perceptible indication of the pulse condition for presentation to a practitioner, such as a visual indicator for presentation via display 630 or an auditory indicator for emitting via a speaker integrated with or connected to computing device 628 (not shown in FIG. 6).

In a further embodiment, as each patient-wearable device is activated and placed for use on patient 650, any additional sensor data generated thereby is sent to computing device 628, which combines it with other sensor data and interprets the combined sensor data to generate and present a result (e.g., a perceptible indication of a pulse condition of patient 650). The sensor data may be transmitted to computing device 628 by each patient-wearable device may include an identifier of the patient-wearable device from which it originated. Computing device 628 may be configured to dynamically switch from operating with a single patient wearable-device to operating with multiple patient-wearable devices as new streams of sensor data are received.

In one embodiment of the system shown in FIG. 6, the patient-wearable devices all possess the same type of sensor and thus generate the same type of sensor data. However, in an alternate embodiment, the patient-wearable devices possess different types of sensors and thus generate different types of sensor data. For example, some of the patient-wearable devices shown in FIG. 6 may include an IMU but not an acoustic sensor, while other ones of the patient-wearable devices shown in FIG. 6 may include an acoustic sensor but not an IMU. Furthermore, some of the patient-wearable devices shown in FIG. 6 may include sensors for detecting qualities of the patient other than the presence of a pulse (e.g., blood pressure, blood sugar, blood oxygen, echocardiogram, body temperature, respiratory rate, blood flow rate), while other ones of the patient-wearable devices shown in FIG. 6 may not include such sensors. In further accordance with such embodiments, each patient-wearable device may include an identifier of the type of sensor used to generate the sensor data when transmitting the sensor data to computing device 628.

In one embodiment of system 600, each of the patient-wearable devices is capable of one-way communication with computing device 628 and utilizes such one-way communication to send sensor data 638 thereto. In an alternate embodiment, each of the patient-wearable devices is capable of two-way communication with computing device 628. For example, in accordance with such an embodiment, each of the patient-wearable devices is capable of sending sensor data 638 to computing device 628 and is also capable of receiving device control information 640 therefrom. Different ones of the patient-wearable devices may utilize different frequency bands and/or different time periods to communicate with computing device 628 so as to avoid interference.

Device control information 640 may comprise any information sent by computing device 628 to control the operation of any one of the patient-wearable devices. For example, in an embodiment, the patient-wearable devices may be designed to operate in a sleep mode (e.g., low power consumption mode) to preserve power of a battery included therein, thereby enabling the device operate over a longer period of time. For example, during sleep mode, the generation and/or transmission of sensor data may be disabled. In further accordance with such an embodiment, computing device 628 may send a “sleep” command to any one of the patient-wearable devices to place the device into sleep mode and also send a “wake” command to the device to cause it to resume generating and transmitting sensor data. Such a feature may be particularly useful for codes, which can last from 30 to 45 minutes, to cause pulse checks to occur every two to three minutes with each active pulse check lasting up to 30 seconds. One or more patient-wearable devices may be wakened from sleep mode to perform the pulse check, and then placed back into sleep mode when the pulse check is finished.

In another embodiment, device control information 640 may include information that can assist with system function monitoring. For example, device control information 640 may include an error message that indicates that there is an error in a data stream received from a patient-wearable device, a lack of a data stream altogether, or some other issue, such that the device can take some action to rectify the issue or notify a user thereof. For example, in an embodiment in which the patient-wearable device includes one or more LED indicators, the receipt of such an error message may cause the patient-wearable device to utilize such LED indicator(s) to signal that an error condition exists.

In certain embodiments, the processing of sensor data, detection of a pulse condition, and generation of an indicator thereof may all be performed by a single patient-wearable device without the need for an external computer. In other embodiments, each patient-wearable device may include the capacity for processing sensor data as well as the ability to perform multi-way communication with one or more external computers or devices. The external computers or devices may themselves be other patient-wearable devices. In such a case, any computation necessary to process sensor data and/or determine a pulse condition may take place in a distributed manner, occurring across all the patient-wearable devices, with communication happening between them, and one or multiple ones of the patient-wearable devices may present an indication of a determined pulse condition. The determination of which patient-wearable devices perform which functions may be negotiated dynamically amongst the patient-wearable devices. Alternatively, a single patient-wearable device may be determined to be a master or primary device and the other patient-wearable devices may be determined to be slave or secondary devices, and the master/primary device may determine which slave/secondary devices perform which functions.

In a further embodiment, a particular one of the patient-wearable devices (e.g., a primary or master device) attached to a patient may collect sensor data from other patient-wearable devices attached to the patient via a short-range wireless communication protocol (e.g., Bluetooth®) or even through wired connections thereto. The particular one of the patient-wearable devices may then transmit the collected sensor data along with its own sensor data to an external computer (e.g., computing device 628) using a long-range wireless communication protocol (e.g., WiFi®), and the external computer can process the sensor data to detect one or more pulse conditions and generate indicator(s) thereof.

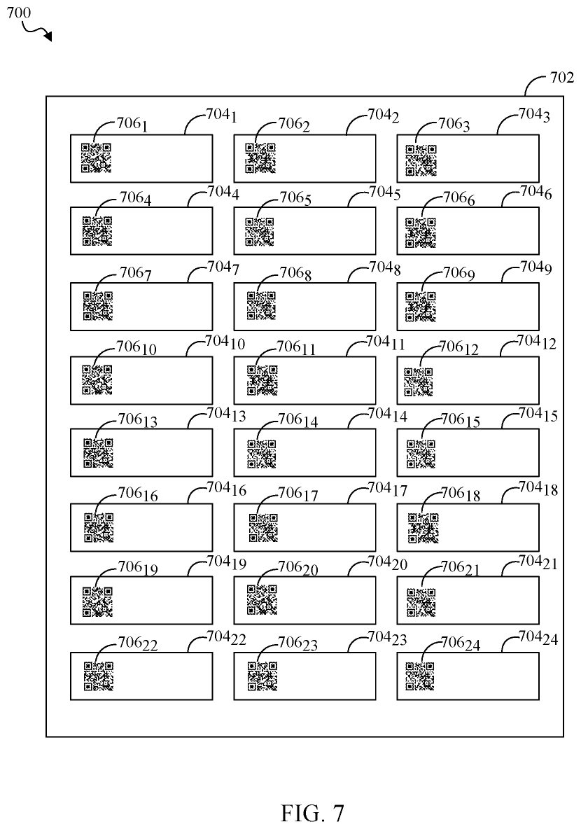

Prior to application to a patient, patient-wearable devices (such as device 100) may be stored adhesive-side down on a suitable substrate, such as a sheet or roll. For example, FIG. 7 depicts a sheet 700 of twenty-four patient-wearable devices 704₁-704₂₄ in accordance with an embodiment. It will be readily understood that a different number of devices may be accommodated on a sheet depending on the size and shape of the sheet and of the respective devices. Sheet 700 comprises a backing sheet 702. Devices 704₁-704₂₄ may be secured adhesive-side down to backing sheet 702 and can be removed therefrom as desired for application to the body of a patient. Backing sheet 702 may be formed from plastic, from paper coated with a suitable release agent (e.g., silicone, polyethylene terephthalate (PET) plastic resin, or polypropylene plastic resin), or from any other materials suitable for facilitating easy removal of devices 704₁-704₂₄ therefrom with the corresponding adhesive layer substantially intact.

As shown in FIG. 7, each device 704₁-704₂₄ has a corresponding (e.g., unique) identifier 706₁-706₂₄ printed thereon. In the example of FIG. 7, the identifier comprises a barcode but any type of identifier may be used. Such identifier can be scanned prior to, during, or after use of a patient-wearable device to associate it with a particular patient. If the patient-wearable device includes an internal battery, a battery preservation mechanism can be utilized while the device sits dormant. For example, as a patient-wearable device is removed from its respective sheet, roll, or other storage location, a small tab can be pulled on the device that allows the battery to make contact with the rest of the device, powering it on. In another embodiment, the act of separating the patient-wearable device from the sheet or roll or scanning the corresponding identifier has the effect of powering on the device. Still other methods and mechanisms for powering on a patient-wearable device may be used.

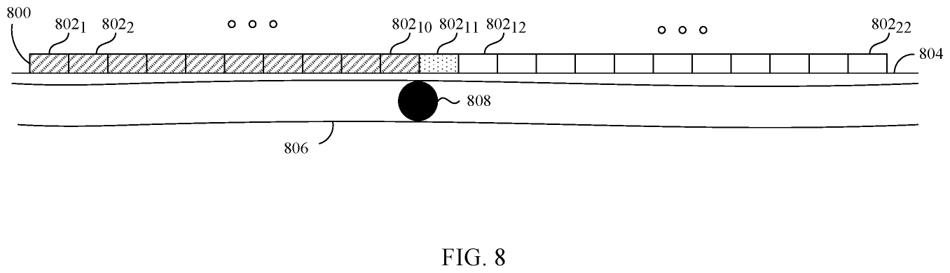

In certain embodiments, a patient-wearable device may provide a comparative grade of pulse strength along its length or on its structure. For example, FIG. 8 illustrates a side view of a patient-wearable device 800 in accordance with such an embodiment. As shown in FIG. 8, device 800 is applied to the skin 804 of a patient at a location along an artery 806 where an arterial occlusion 808 exists. In the example of FIG. 8, device 800 comprises twenty-two sensing modules 802₁-802₂₂, although a smaller or greater number of modules may be included as deemed necessary or desirable. Each of the sensing modules 802₁-802₂₂ may include one or more sensors to generate sensor data, a processing unit (e.g., microcontroller, microprocessor, DSP and/or ASIC) to process the sensor data and determine a pulse strength therefrom, and a visual indicator (e.g., LED indicator or the like) of the determined pulse strength. However, in an alternate embodiment, a sensing module may utilize a single sensor and/or processing unit for driving multiple visual indicators across its length. For example, a detection area of a sensor may be such that it can drive multiple visual indicators along its length to indicate pressure change across the span from beginning to end of the sensor.

In the example shown in FIG. 8, the visual indicators of sensing modules 802₁₂-802₂₂ on device 800 proximal to occlusion 808 signify detection of a pulse (e.g., by showing a relatively bright illumination or by illuminating a green light) in the patient, while the visual indicators of sensing modules 802₁-802₁₀ associated with the distal aspect of artery 806 signify no pulse (e.g., by showing no illumination or by illuminating a red light). The transition between the two suggests the location over the occlusion, which is further be signified by a visual indicator of sensing module 802₁₁ (e.g., by showing an intermediate level of illumination or by illuminating a yellow light). This embodiment of FIG. 8 thus allows for obstruction location detection by a medical provider.

As discussed above in reference to FIG. 1, patient-wearable device 100 may comprise an adhesive layer 112 that enables device 100 to be affixed to the body (e.g., the skin) of a patient. Such adhesive layer may comprise, for example and without limitation, a ready-for-use adhesive pad or film or a replaceable adhesive pad or film that may be attached to the skin or attached to device 100 prior to application to a patient. In the embodiment of FIG. 1, adhesive layer 112 is attached to the bottom of device 100, which may be contacting PCB 102, to the bottom of a base sheet that includes PCB 102 or to which PCB 102 itself is connected, or to some other component of device 100 that can contact the body of a patient. However, this example is not intended to be limiting. An adhesive layer may be connected to a patient-wearable device in a variety of different ways to support affixing the device to the patient.

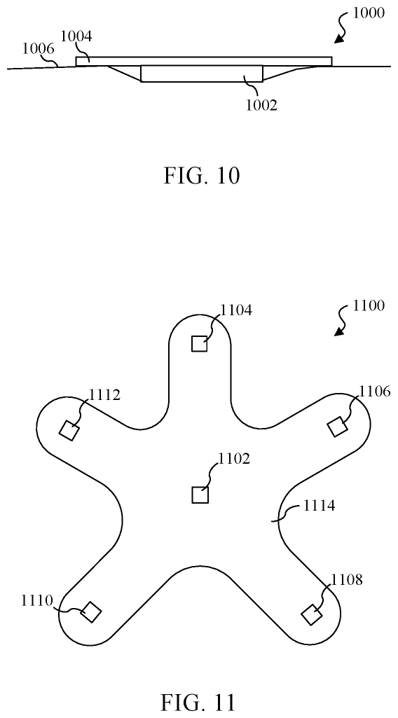

By way of example, FIG. 10 depicts a side view of a patient-wearable device 1000 that includes a housing 1002 and an adhesive layer 1004 that is attached to and extends laterally outward from a top side of housing 1002. With respect to the embodiment of FIG. 10, the “top side” of housing 1002 is the side of housing 1002 that faces away from the body of the patient when device 1000 is attached thereto, while the “bottom side” of housing 1002 is the side of housing 1002 that faces toward the body of the patient when device 1000 is attached thereto. Housing 1002 is intended to represent any structure that houses, encapsulates, covers or supports a PCB as well as electronics and an antenna that are disposed on such PCB (such as PCB 102, electronics 106 and antenna 108 as described above in reference to FIG. 1) and that operate in a manner described above to facilitate the detection of a pulse condition of a patient.

As further shown in FIG. 10, at least a portion of adhesive layer 1004 that extends outward from the top side of housing 1002 may be affixed to a body 1006 of a patient in such a manner that housing 1002, or some portion of device 1000 contained in housing 1002, is pushed, to at least some degree, into the surface of the skin of the patient while device 1000 is attached thereto. Pushing housing 1002 into the surface of the skin of the patient may have a beneficial effect, for example, by bringing the sensors of device 1000 closer to the blood vessels of the patient or by physically separating certain sensors from sources of stimuli, thereby enhancing the ability of those sensors to detect a pulse or subpulse of the patient. Also, because housing 1002 may essentially be sealed between adhesive layer 1004 and the skin of the patient when device 1000 is attached thereto, housing 1002 may be rendered less susceptible to being jostled or moved due to external forces (e.g., someone or something brushing up against the body of the patient). Likewise, because housing 1002 may essentially be sealed between adhesive layer 1004 and the skin of the patient when device 1000 is attached thereto, sensors present on or within housing 1002 may be rendered less susceptible to ambient stimuli in the environment around the patient if adhesive layer 1004 has stimulus-dampening (e.g., in the case of an acoustic sensor, sound-absorptive and/or sound-reflective) characteristics. That is to say, when adhesive layer 1004 has certain stimuli-dampening characteristics, the encapsulation of such sensors between adhesive layer 1004 and body 1006 of the patient may help to isolate such sensors from external stimuli sources, or at least mitigate the impact of such external stimuli sources on the sensors.

Although the foregoing describes the use of adhesive layer 1004 to push housing 1002 into the surface of the skin of the patient, it is also noted that the attachment of adhesive layer 1004 to the surface of the skin of the patient may additionally or alternatively cause the skin that surrounds housing 1002 to be pulled upward, achieving a similar encapsulating effect. Furthermore, factors other than the use of adhesive layer 1004 may cause the patient-wearable device to be pushed into the surface of the skin of the patient, such as the weight or firmness of device 1000 itself. For example, depending upon the choice of materials, the weight of an overlay or housing that covers or encapsulates the PCB, electronics, and antenna of the device may have the effect of causing the device to be pushed into the surface of the skin of the patient when the patient-wearable device is attached thereto.

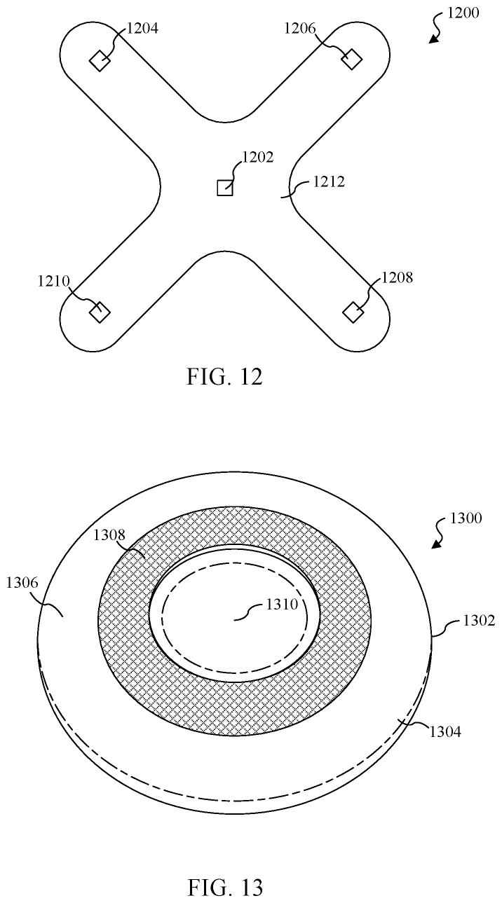

An adhesive layer that is used to affix a patient-wearable device to a body of a patient may have any of a variety of shapes and be of any of a variety of sizes appropriate for a particular location on the body or for a specific sensor combination. For example, FIG. 11 depicts a patient wearable device 1100 that includes a star-shaped adhesive layer 1114, while FIG. 12 depicts a patient wearable device 1200 that includes an X-shaped adhesive layer 1212. Generally speaking, utilizing an adhesive layer with greater surface area and/or multiple arms may increase the size and/or the number of the points of contact between the adhesive layer and the body of the patient and/or facilitate a desired distance between sensors within device 100, thereby improving the ability of device 100 to achieve its intended function. Furthermore, the shape and size of the adhesive layer may be selected based on suitability for attachment to a particular body part of the patient. For example, a star shape or X shape may be particularly suitable for attachment to a patient's chest (e.g., over the patient's heart and ribs), while a band, cuff, elongated rectangle, square, circle, or irregular shape may be particularly suitable for attachment to a patient's limb.

Although the patient-wearable devices of FIGS. 11 and 12 each include an adhesive layer that comprises a plurality of “arms” of similar length, this need not be the case and different arms of each device's adhesive layer may be of different lengths.

Furthermore, in certain embodiments, a plurality of sensing modules may be attached to different portions of a single adhesive layer, such that the plurality of sensing modules can easily be attached to the body of a patient through the application of the single adhesive layer. For example, as shown in FIG. 11, patient-wearable device 1100 comprises six sensing modules 1102, 1104, 1106, 1108, 1110 and 1112, each of which is attached to a different portion of adhesive layer 1114 and, as shown in FIG. 12, patient-wearable device 1200 comprises five sensing modules 1202, 1204, 1206, 1208 and 1210, each of which is attached to a different portion of adhesive layer 1212. Each of the aforementioned sensing modules may include one or more sensors to generate sensor data and a processing unit (e.g., microcontroller, microprocessor, DSP and/or ASIC) to process the sensor data and determine a pulse strength therefrom. Each of the aforementioned sensing modules may also include an indicator (e.g., a visual indicator such as an LED) that indicates the determined pulse strength and/or a wired or wireless interface for communicating the determined pulse strength within or between devices.

The plurality of sensing modules of patient-wearable device 1100 of FIG. 11 or the plurality of sensing modules of patient-wearable device 1200 of FIG. 12 may operate in a like manner to the plurality of patient-wearable devices of system 600 shown in FIG. 6, as previously described. For example, each of the sensing modules may be capable of transmitting sensor data to a computing device and/or receiving device control information from such computing device via a wired or wireless connection Likewise, the plurality of sensing modules may operate to concurrently detect pulse conditions at different body locations which, as discussed above in reference to FIG. 6, may be particularly beneficial in situations of cardiac arrest, other critical conditions for which rapid pulse detection (or lack thereof) is pivotal, and for monitoring following certain invasive procedures. In certain embodiments, the different sensing modules may be capable of communicating with each other, which may facilitate data sharing amongst the sensing modules as well as distributed processing scenarios.

Different ones of the sensing modules of patient-wearable device 1100 of FIG. 11 or different ones of the sensing modules of patient-wearable device 1200 of FIG. 12 may possess the same type of sensor or may possess different types of sensors. For example, sensing module 1202 of patient-wearable device 1200 of FIG. 12 may include an ECG sensor while the other sensing modules of patient-wearable device 1200 do not. In further accordance with such an example, when patient-wearable device 1200 is attached to the body of a patient, it may be attached in such a manner that sensing module 1202 (or any of sensing modules 1204, 1206, 1208 or 1210) is placed at or near the Point of Maximal Impulse (PMI) on the chest of the patient. This will have the effect of improving the performance of the ECG sensor. As discussed elsewhere herein, the ECG data collected by sensing module 1202 may then be shared with and used by any one or more of the sensing modules or an external computing device to filter data collected by other types of sensors, such as an acoustic sensor or an IMU. For example, sensor data obtained by an acoustic sensor or an IMU may be filtered in at least a time domain by accounting for data that deviates from an electrical signal detected using the aforementioned ECG sensor.

In certain embodiments of patient-wearable device 1100 and patient-wearable device 1200, the adhesive layer is attached to the bottom of the sensing modules such that the adhesive layer will be between the sensing modules and the body of the patient when the device is connected to the body of the patient. In alternate embodiments of patient-wearable device 1100 and patient-wearable device 1200, the adhesive layer is attached to the top of the sensing modules such that the sensing modules will be between the adhesive layer and the body of the patient when the device is connected to the body of the patient. In such an embodiment, the attachment between the adhesive layer and the skin of the patient may have the effect of pushing the sensing modules into the skin of the patient, which may have the beneficial effect of bringing the sensors closer to the blood vessels of the patient and also, when the adhesive layer has stimuli-barring characteristics, isolating any sensors of the sensing modules from external stimuli, as discussed above in reference to the embodiment of FIG. 10. In still further embodiments of patient-wearable device 1100 and patient-wearable device 1200, the adhesive layer may be attached to the bottom of some of the sensing modules and to the top of other ones of the sensing modules such that some of the sensing modules will be above the adhesive layer and other sensing modules will be below the adhesive layer and in contact with the body of the patient when the device is connected to the body of the patient.

Although the embodiments of FIGS. 11 and 12 each have one sensing module at the center of the adhesive layer and one sensing module at or near the end of each arm emanating from the center of the adhesive layer, this need not be the case and the sensing modules may instead be placed at any location with respect to the adhesive layer. For example, in alternative embodiments, a cluster of sensing modules may be located in central part of the adhesive layer and the arms of the adhesive layer may be used only to connect the device to the body of the patient. In other alternative implementations, for example, patient-wearable devices 1100 or 1200 (or any other embodiment described herein) may contain as few as a single sensor module located thereon.

FIG. 13 depicts a top and side perspective view of a patient-wearable device 1300 for detecting a pulse condition of a patient in accordance with a further embodiment. As shown in FIG. 13, device 1300 comprises a housing 1302 that houses, encapsulates, covers or supports various components of device 1300 that will be described in more detail below. In embodiments, housing 1302 is formed from a material suitable for application on the skin of a patient such as, for example, silicone or a silicone-like material such as thermoplastic elastomer (TPE), thermoplastic rubber (TPR), or thermoplastic polyurethane (TPU), although other materials may be used.

As further shown in FIG. 13, housing 1302 comprises a top side 1304 that itself includes an outer circumferential portion 1306, an inner circumferential portion 1308, and a central depression 1310. Central depression 1310 may be formed from a same or different material than the rest of housing 1302. For example, central depression 1310 may be formed from a transparent or quasi-transparent material (e.g., transparent or translucent silicone or plastic) such that certain visual indicators (e.g., LEDs) disposed within housing 1302 will be visible through central depression 1310. Central depression 1310 may be molded or otherwise permanently affixed to top side 1304 of housing 1302. Alternatively, central depression 1310 may comprise a removable cap (e.g., a press fit cap, a screw on cap, a hinged cap, or the like) that can be connected to and disconnected from top side 1304 of housing 1302. In still further embodiments, central depression 1310 may simply comprise an opening in the center of top side 1304 of housing 1302 via which various internal components of device 1300 may be visible or accessible, or central depression 1310 may be omitted entirely.

Inner circumferential portion 1308 may have a rough or uneven surface that makes device 1300 easier to grip and hold onto while outer circumferential portion 1306 may have a smoother surface. This may be particularly helpful when housing 1302 is formed from silicone, which can be slippery, and/or if device 100 becomes wet due to the presence of blood or other liquids. It will be appreciated that different surface qualities of housing 1302 may also cause diffuse refraction of light from light sources from within device 100, which can make visual indicators visible within depression 1310 or elsewhere on housing 1302 easier to see. In alternate embodiments, additional or different portions of top side 1304 of housing 1302 may be textured Likewise, some or all of a circumferential edge or a bottom of housing 1302 may be textured depending upon the implementation. It will be further appreciated that while housing 1302 of FIG. 13 comprises a top side 1304 that is dome-shaped, housing 1302 and top side 1304 may be flatter or more rounded in shape to meet the desired configuration for an application or location on the body of the patient.

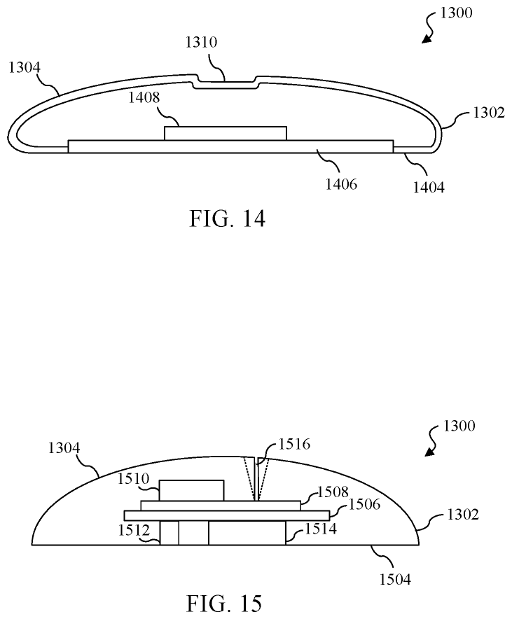

FIG. 14 depicts a cross-sectional side view of device 1300 in accordance with one embodiment. In the embodiment of FIG. 14, device 1300 includes a PCB 1406 (e.g., a single-sided PCB) and a bottom side 1404 of housing 1302 comprises a bottom side of PCB 1406. Furthermore, various electronic components 1408 are connected to a top side of PCB 1406. These electronic components 1408 may include components described herein (e.g., components described above in reference to FIG. 4), such as one or more sensors, one or more passive electronic components, a battery, a microcontroller, and an antenna, and such electronic components 1408 may operate in a manner described above to facilitate the detection of a pulse condition of a patient.

In an embodiment, housing 1302 of device 1300 may be formed by depositing or molding a material (e.g., silicone) on top of or around PCB 1406 and electronic components 1408. Housing 1302 may thus be substantially solid throughout. In an alternative implementation, an internal cavity may be created between the top of PCB 1406/electronics 1408 and an inner side of top 1304 of housing 1302 and such cavity may comprise a material that alters the performance of the sensors.

In the embodiment shown in FIG. 14, the material of housing 1302 that is disposed above electronic components 1408 may act as an insulating layer with respect to various sensors included in electronic components 1408. For example, in an embodiment in which electronic components 1408 include an IMU, the material of housing 1302 may insulate the IMU from forces that originate outside of a patient's body that might otherwise be sensed, or more strongly sensed, by the IMU. As another example, in an embodiment in which electronic components 1408 include an acoustic sensor, the material of housing 1302 may insulate the acoustic sensor from sound waves that emanate from sources outside of a patient's body that might otherwise be sensed, or more strongly sensed, by the acoustic sensor. For example, the material of housing 1302 may have sound-barring (e.g., sound-reflecting and/or sound-absorbing qualities) characteristics that may help insulate the acoustic sensor from external sources of noise.

FIG. 15 depicts a cross-sectional side view of device 1300 in accordance with another embodiment. In the embodiment shown in FIG. 15, device 1300 includes a double-sided PCB 1506 having a top side to which electronic components 1508 and 1510 are attached and a bottom side to which electronic components 1512 and 1514 are attached. The electronic components collectively attached to either side of PCB 1506 may include components described herein (e.g., components described above in reference to FIG. 4), such as one or more sensors, one or more passive electronic components, a battery, a microcontroller, and an antenna, and such components may operate in a manner described above to facilitate the detection of a pulse condition of a patient.

In an embodiment, housing 1302 of device 1300 may be formed by depositing or molding a material (e.g., silicone) around PCB 1406 and electronic components 1508, 1510, 1512 and 1514, such that body-facing sides of electronic components 1512 and 1514 are substantially flush with and essentially form a part of a bottom 1504 of housing 1302. In an implementation in which electronic components 1512 and 1514 include sensors (e.g., IMUs or acoustic sensors), such a design may beneficially position such sensors as close to the body of the patient as possible and minimize or remove any structural barriers (e.g., PCB 1506 or a portion of housing 1302) from between such sensors and the body of the patient, all of which may improve the ability of such sensors to detect a pulse condition of the patient.

As further shown in FIG. 15, housing 1302 may comprise or form a channel 1516 that extends from electronics components 1508 on the top side of PCB 1506 to top side 1304 of housing 1302. When the embodiment of FIG. 15 is viewed from above, channel 1516 may appear as a hole or a cone. Electronic components 1508 located at or near the bottom of channel 1516 may comprise a first acoustic sensor (e.g., a PCB-based low-powered MEMs acoustic sensor). The presence of channel 1516 may enable such first acoustic sensor to sense, or more strongly and/or accurately sense, sound waves generated by sources in an environment around device 1300 and external to the body of the patient. By operating in this manner, the first acoustic sensor can identify one or more background noise signals. Such background noise signal(s) may then be used to filter or correct audio signals captured by one or more other acoustic sensors within device 1300 and/or within other devices that are directly or indirectly communicatively connected to device 1300.

For example, the embodiment of FIG. 15 may further include one or more acoustic sensors connected to PCB 1506 (e.g., as part of electronic components 1512 or 1514) that are positioned to receive stimuli from the body of the patient when device 1300 is connected thereto. In accordance with such an embodiment, PCB 1506 and housing 1302 may act as acoustic barriers that help isolate individual acoustic sensor(s) from one another and from other sources of stimuli. Thus, such additional acoustic sensor(s) may obtain a cleaner (e.g., more noise-free) audio signal for detecting a pulse condition of the patient. Moreover, to the extent audio data captured by any acoustic sensor includes noise components different from the other(s) by type or intensity, such noise components may be partially or substantially removed, filtered or otherwise accounted for as desired.

Although not shown in FIG. 15, in certain embodiments in which a second acoustic sensor is not flush with bottom 1504 of housing 1302 of device 1300, a second channel may be formed between the second acoustic sensor and bottom 1504 of housing 1302 to enhance the ability of such second sensor to detect a pulse or subpulse of the patient. Furthermore, although the above description mentions the possibility of a single acoustic sensor on either side of PCB 1506, it should be understood that any number of acoustic sensors may be placed on either side of PCB 1506. For example, two or more acoustic sensors may be disposed on the top side of PCB 1506 and used to capture ambient/background noise while two or more acoustic sensors may be disposed on the bottom side of PCB 1506 and used to detect a pulse or subpulse. Additionally, any number of channels may be formed in top 1304 of housing 1302 or bottom 1504 of housing 1302 to improve the performance of such acoustic sensors.

Also, although only a single PCB 1506 is shown in FIG. 15, it is to be understood that any number of PCBs may be present in device 1300. For example, two PCBs may be present (e.g., one affixed at a certain distance atop the other) to provide a four-layer system. The number of PCBs used may be determined based on such factors as the number and size of electronic components to be included in device 1300, the size and shape of device 1300, or the like.

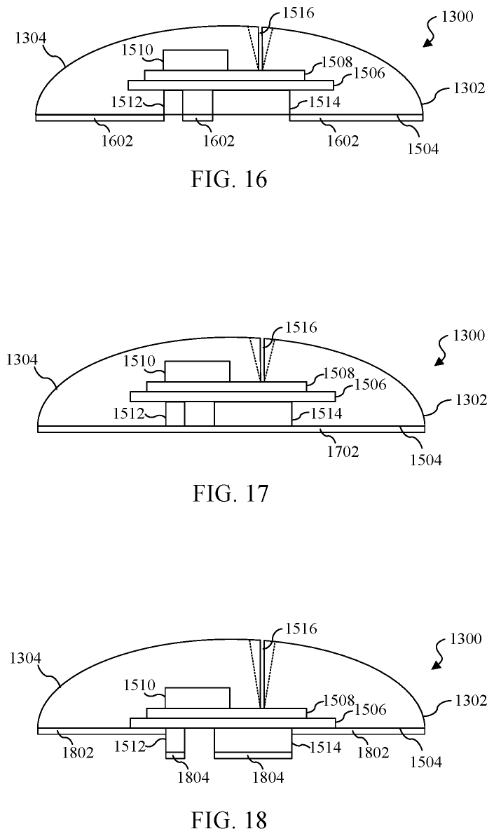

FIG. 16 depicts a cross-sectional side view of a further embodiment of device 1300 that is similar to the embodiment shown in FIG. 15, except that an adhesive layer 1602 has been disposed on or attached to bottom side 1504 of housing 1302. Adhesive layer 1602 may be used to facilitate attachment of device 1300 to the body (e.g., the skin) of a patient and may be implemented in any manner previously described herein, including in any manner previously described with reference to adhesive layer 112 of device 100. In the embodiment of FIG. 16, adhesive layer 1602 only partially covers bottom side 1504 of housing 1302. In particular, adhesive layer 1602 does not cover the portions of bottom side 1504 of housing 1302 beneath electronics components 1512 and 1514. Such a design may be deemed desirable if adhesive layer 1602 has one or more characteristics that may impede or degrade the functioning of electronics components 1512 and 1514. For example, such a design may be deemed desirable if the presence of adhesive layer 1602 negatively impacts the performance of (e.g., attenuates a signal sensed by) an acoustic sensor or IMU within electronic components 1512 and 1514.

FIG. 17 depicts a cross-sectional side view of a further embodiment of device 1300 that is similar to the embodiment shown in FIG. 15, except that an adhesive layer 1702 has been disposed on or attached to bottom side 1504 of housing 1302. Adhesive layer 1702 may be used to facilitate attachment of device 1300 to the body (e.g., the skin) of a patient and may be implemented in any manner previously described herein, including in any manner previously described with reference to adhesive layer 112 of device 100. In the embodiment of FIG. 17, adhesive layer 1702 covers all or substantially all of bottom side 1504 of housing 1302. Such a design may be deemed desirable if adhesive layer 1602 does not have any characteristics that may impede or degrade the functioning of electronics components 1512 and 1514, or if adhesive layer 1602 has one or more characteristics that may improve the functioning of electronics components 1512 and 1514. For example, such a design may be deemed desirable if the presence of adhesive layer 1702 improves the performance of (e.g., amplifies a signal sensed by) an acoustic sensor or IMU within electronic components 1512 and 1514.

FIG. 18 depicts a cross-sectional side view of a further embodiment of device 1300 that is similar to the embodiment shown in FIG. 15, except that in this embodiment the internal components have been shifted downward, such that electronic components 1512 and 1514 protrude outward from bottom side 1504 of housing 1302. A first adhesive layer 1802 may be disposed on or attached to bottom side 1504 of housing 1302, while a second adhesive layer 1804 may optionally be disposed on or attached to electronic components 1512 and 1514. These adhesive layers may be used to facilitate attachment of device 1300 to the body (e.g., the skin) of a patient and may be implemented in any manner previously described herein, including in any manner previously described with reference to adhesive layer 112 of device 100. When the embodiment of device 1300 shown in FIG. 18 is attached to the body of a patient, the particular configuration of components will cause electronic components 1512 and 1514 to be pushed deeper into the skin of the patient. When electronic components 1512 and 1514 comprise sensors, this may have the beneficial effect of bringing the sensors closer to the blood vessels of the patient, thereby enhancing the ability of those sensors to detect a pulse or subpulse of the patient.

In embodiments discussed above with respect to FIGS. 6, 11 and 12, different patient-wearable devices or different sensing modules may be attached to different locations on a body of a patient and concurrently utilized to detect a pulse condition of the patient. As also noted above, the different patient-wearable devices or different sensing modules may comprise different sensors. For example, a first patient-wearable device or sensing module attached to a first location on a body of a patient may include a first type of sensor but not a second type of sensor, whereas a second patient-wearable device or sensing module attached to a second location on the body of the patient may include the second type of sensor but not the first type of sensor. Possible types of sensors that may be included within a patient-wearable device or sensing module may include but are not limited to: an accelerometer, a gyroscope, a magnetometer, an IMU (which may itself comprise one or more of an accelerometer, a gyroscope or a magnetometer), an acoustic sensor, an ECG sensor, a carbon dioxide (CO₂) sensor, a blood oxygen (SpO₂) sensor, or a sensor that is capable of detecting one or more of blood pressure, blood sugar, blood pH, body temperature, respiratory rate, blood flow rate, magnetic fields, or the like.

In certain embodiments, each of a plurality of patient-wearable devices or sensing modules may include the same plurality of sensor types, but individual ones of the devices/modules may be controlled to activate a different subset of the plurality of sensor types. That is to say, each device/module may be individually controlled to activate or deactivate different ones of the plurality of sensor types included therein. By way of example, assume that each of the plurality of devices/modules includes an IMU, an acoustic sensor and an ECG sensor. Furthermore, assume that a respective one of these devices/modules have been attached to the following locations on the body of the patient: over the heart, on the right wrist, on the left wrist, on the right ankle, and on the left ankle In accordance with such a scenario, the device/module over the heart may be controlled such that only the ECG sensor is active and the other sensors, such as IMUs or acoustic sensors, are inactive, the devices/modules on the wrist may be controlled such that various combinations of IMUs and/or acoustic sensors are active and the ECG sensor is inactive, and the devices/modules on the ankles may be controlled such that the same or different combinations of IMUs and/or acoustic sensors as in the devices/modules on the wrists are active and the ECG sensor is inactive. Of course, this is only one example and a wide variety of different sensor types and selective activation/deactivation control schemes may be used.

Control of a patient-wearable device or sensing module for the purposes of selectively activating or deactivating one or more sensors included therein may be achieved in a variety of ways. For example, if the patient-wearable device or sensing module comprises a wired or wireless interface, then the device/module can receive commands from an external device (e.g., from a computing device such as computing device 628 of FIG. 6 or from another patient-wearable device or sensing module) and then execute such commands to cause one or more sensors to be activated or deactivated. As another example, the patient-wearable device or sensing module may comprise one or more mechanical user interface elements (e.g., buttons, toggles, switches, or the like) that a user may interact with to selectively activate or deactivate particular sensors on the device/module. In an alternative embodiment, individual patient-wearable devices or sensing modules may be oriented (by user input, location-specific device selection, barcode, or some other means) according to its respective location on the body of the patient, such as the carotid artery, which may dictate which sensor combination is utilized in a predefined manner Such location-specific orientation may, for example, aid data processing and filtering of incoming location-specific stimuli.

An approach in which all of the patient-wearable devices or sensing modules include the same complement of sensors but that allows for sensors to be activated or deactivated on a per device/module basis can be beneficial in that only one version of the device/module need be produced and each device/module can be applied to any location on the body of the patient without regard for which sensors are on board. In contrast, in accordance with an alternate approach in which different patient-wearable devices or sensing modules actually include different sensors, multiple versions of the devices/modules must be produced and certain versions of the devices/modules may be targeted to particular body locations where certain sensors are desired or most effective. Benefits may be achieved from either approach and may include one or more of the following: ease of use; cheaper manufacturing costs per device/module; superior data collection due to less interference between onboard sensors; more optimal placing of sensors; reduced power consumption per device/module; and increased flexibility in trace geometry and/or overall PCB design.

It is noted that some embodiments may combine the foregoing approaches. That is to say, in some embodiments, a plurality of patient-wearable devices or sensing modules may all share a common set of sensor types that may be selectively activated/deactivated but they may also include different sensor types as well.

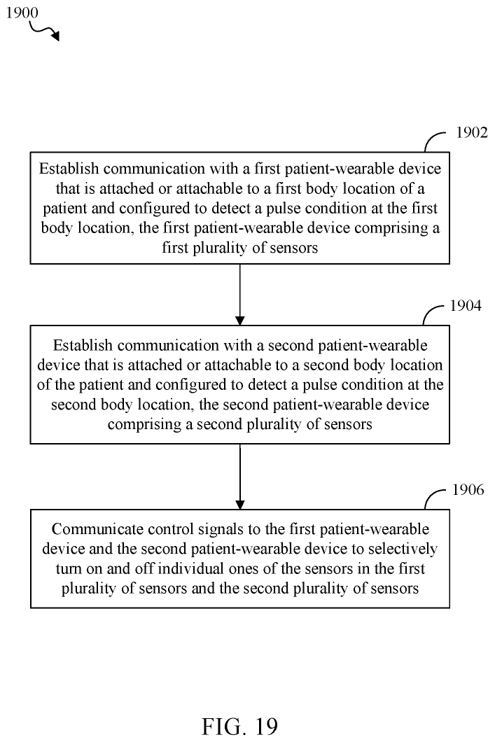

FIG. 19 depicts a flowchart 1900 of a method for selectively activating/deactivating sensors of a plurality of patient-wearable devices that are attached or attachable to different locations on a body of a patient, in accordance with an embodiment. The method of FIG. 19 may be implemented, for example, by processing unit 634 of system 600 which, as discussed above in reference to FIG. 6, may be communicatively connected to multiple patient-wearable devices 602, 604, 606, 608, 610, 612, 614, 616, 618, 620, 622, 624, 626 that are concurrently attached to different locations on the body of patient 650. However, this example is not intended to be limiting and the method of flowchart 1900 may be implemented by any processing unit that is capable of connecting to and communicating with multiple patient-wearable devices, wherein each of the patient-wearable devices comprises a plurality of sensors. Such processing unit may, for example, be separate from and communicatively connected to each of the patient-wearable devices (as is the case in system 600 of FIG. 6), or such processing unit may form part of one or more of the patient-wearable devices. Still further, the steps of flowchart 1900 may be performed in a distributed manner by multiple processing units.

Furthermore, although the method of flowchart 1900 refers to a plurality of patient-wearable devices that each comprise a plurality of sensors, it is to be understood that the method can also be applied to a plurality of sensing modules (e.g., the plurality of sensing modules present in the respective embodiments of FIG. 8, FIG. 11 or FIG. 12) when such sensing modules comprise a plurality of sensors.

As shown in FIG. 6, the method of flowchart 1900 begins at step 1902 in which the processing unit establishes communication with a first patient-wearable device that is attached or attachable to a first body location of a patient and configured to detect a pulse condition at the first body location, wherein the first patient-wearable device comprises a first plurality of sensors. For example, the first patient-wearable device may a first one of patient-wearable devices 602, 604, 606, 608, 610, 612, 614, 616, 618, 620, 622, 624, 626 which is attached or attachable to a particular location on the body of patient 650 as discussed above in reference to FIG. 6. The first patient-wearable device may be configured to detect a subpulse condition at the respective body location. The processing unit may establish communication with the first patient-wearable device using one or more of a wireless communication link (e.g., a Bluetooth®, Wi-Fi® or other RF communication link) or a wired communication link (e.g., a USB or other wired communication link)

At step 1904, the processing unit establishes communication with a second patient-wearable device that is attached or attachable to a second body location of the patient and configured to detect a pulse condition at the second body location, wherein the second patient-wearable device comprises a second plurality of sensors. For example, the second patient-wearable device may be a second one of patient-wearable devices 602, 604, 606, 608, 610, 612, 614, 616, 618, 620, 622, 624, 626 which is attached or attachable to a particular location on the body of patient 650 as discussed above in reference to FIG. 6. The second patient-wearable device may also be configured to detect a subpulse condition at the respective body location. The processing unit may establish communication with the second patient-wearable device using one or more of a wireless communication link (e.g., a Bluetooth®, Wi-Fi® or other RF communication link) or a wired communication link (e.g., a USB or other wired communication link)

In certain embodiments, the first plurality of sensors and the second plurality of sensors include a same variety of sensor types. For example, the first plurality of sensors may include at least a first IMU and a first acoustic sensor and the second plurality of sensors may include at least a second IMU and a second acoustic sensor. In further accordance with such an embodiment, the first plurality of sensors and the second plurality of sensors may further include: a first ECG sensor and a second ECG sensor, respectively; a first carbon dioxide sensor and a second carbon dioxide sensor, respectively; and/or a first blood oxygen sensor and a second blood oxygen sensor, respectively. However, these are only non-limiting examples, and the first plurality of sensors and the second plurality of sensors may both include still other sensor types.

At step 1906, the processing unit communicates control signals to the first patient-wearable device and the second patient-wearable device to selectively turn on and off individual ones of the sensors in the first plurality of sensors and the second plurality of sensors. The processing unit may selectively turn on or off the individual ones of the sensors in the first plurality of sensors and the second plurality of sensors based on one or more of: body locations to which the first patient-wearable device and the second patient-wearable device are or will be respectively attached; one or more factors associated with the environment of the patient; or a type of medical procedure that was or will be performed on the patient.

In a case where the selective activation/deactivation of sensors is performed based on the body locations of the first and second patient-wearable devices, the body location information for each patient-wearable device may be determined in a variety of ways. For example, a user may input information (e.g., to computing device 628) that associates an identifier of each patient-wearable device with a particular body location. In accordance with an embodiment such as that described above in reference to FIG. 7, in which a user may scan an identifier (e.g., barcode) of a patient-wearable device to associate the device with a particular patient, the user may also utilize the identifier to associate the device with a particular body location of the patient at the time of device activation or attachment. Such identifier may also include information about which sensors are included within the device. As another example, each patient-wearable device may communicate its own identifier and body location to the processing module via the aforementioned wireless or wired communication links. In such a scenario, each patient-wearable device may be pre-programmed to operate at a particular body location and transmits an indicator of the particular body location to the processing module. Alternatively, each patient-wearable device may be capable of sensing the body location to which it has been attached and transmits this information to the processing module. As yet another example, an external sensing device or system may determine the body locations of the patient-wearable determined by scanning the body of the patient to identify particular patient-wearable devices and the respective body locations to which they are attached and then pass such information to the processing module. However, these are only examples and still other techniques may be used to determine the body location information for each patient-wearable device.

In a case where the selective activation/deactivation of sensors is performed based on one or more factors associated with the environment of the patient, such factor(s) may be determined in a variety of ways. For example, a user may input information regarding such factor(s) (e.g., to computing device 628). As another example, the factor(s) may be determined by one or more of the patient-wearable devices (e.g., based on sensor data captured thereby) and communicated to the processing unit via the aforementioned wireless or wired communication links. As yet another example, an external sensing device or system may determine the factor(s) and then pass such information to the processing module. However, these are only examples and still other techniques may be used to determine the factor(s) associated with the environment of the patient.

In an embodiment, the processing module may operate to selectively turn on all sensors of a first type in the first plurality of sensors and turn off all sensors of the first type in the second plurality of sensors. For example, with reference to an example discussed above, the processing module may turn on an ECG sensor in the first plurality of sensors when the first patient-wearable device is attached to the patient's chest at or near the heart, while turning off the ECG sensor in the second plurality of sensors when the second patient-wearable device is attached to an extremity (e.g., wrist or ankle) of the patient.

In another embodiment, the processing module may operate to selectively turn on all sensors of a first type in the first plurality of sensors and the second plurality of sensors and turn off all sensors of a second type in the first plurality of sensors and the second plurality of sensors. For example, when the patient is in an environment with significant external noise, the processing module may turn off all acoustic sensors in the first plurality of sensors and the second plurality of sensors and turn on all IMUs in the first plurality of sensors and the second plurality of sensors. As another example, when the patient is moving or being moved (e.g., in a moving ambulance), the processing module may turn off all IMUs in the first plurality of sensors and the second plurality of sensors and turn on all acoustic sensors in the first plurality of sensors and the second plurality of sensors.

Various motivations may exist for providing different sensor types at different body locations. For example, having at least an accelerometer at different body locations may be deemed highly beneficial since different body parts may be moving differently. Thus, accelerometer data obtained from multiple locations can be compared and differences due to non-pulse-related motion can be identified and accounted for. Furthermore, ECG data (electrical signals) can be useful for interpreting overall collected data. As another example, in the case of detecting something like an obstructed artery, it may be useful to place devices in multiple locations to identify a potential blockage site and utilize a suite of sensors (e.g., IMU, acoustic sensor and ECG sensor) in those locations.

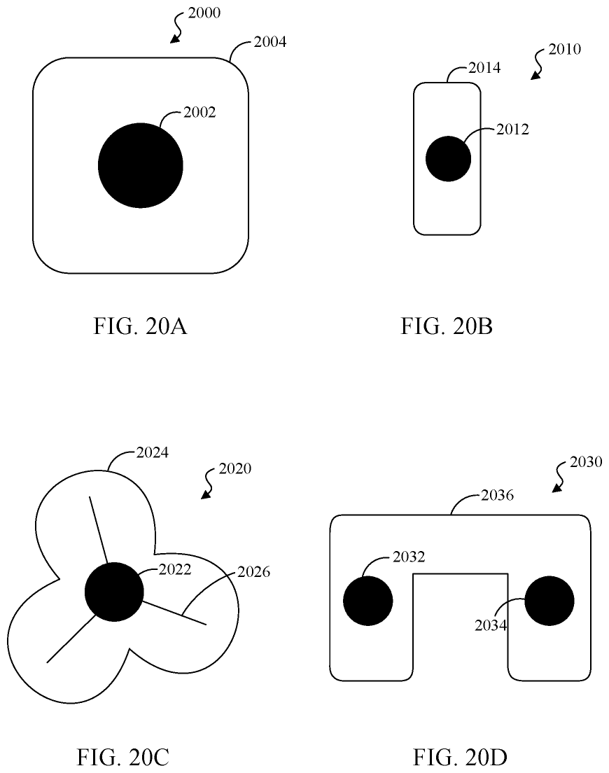

FIGS. 20A, 20B, 20C and 20D illustrate different patient-wearable device configurations that may be suitable for attachment to respective different locations on a body of a patient, in accordance with embodiments. Generally speaking, each of the patient-wearable device configurations includes one or more sensing modules attached to an adhesive layer, wherein the sensing module(s) may be implemented in a like manner to the sensing modules described above in reference to FIG. 8, 11 or 12, although the sensing module(s) may be implemented in other ways as well.

For example, FIG. 20A depicts a patient-wearable device 2000 that has a roughly square shape and that comprises a sensing module 2002 that is attached to an adhesive layer 2004. The configuration shown in FIG. 20A may be particularly suitable for attachment to a thigh of a patient, over or near a femoral artery thereof, or on an inside of a forearm of a patient, over or near a radial artery thereof.

FIG. 20B depicts a patient-wearable device 2010 that has a roughly rectangular shape and that comprises a sensing module 2012 that is attached to a an adhesive layer 2014. The configuration shown in FIG. 20B may be particularly suitable for attachment to a side of a neck of a patient, over a carotid artery thereof.

FIG. 20C depicts a patient-wearable device 2020 that has a roughly trefoil shape and that comprises a sensing module 2022 that is attached to an adhesive layer 2024. As further shown in FIG. 20C, patient-wearable device 2020 further comprises three leads or wires 2026 that emanate outward from sensing module 2022, each of which may terminate at a corresponding electrode, and each of which may be utilized to collect electrical signals for use by an ECG sensor included within sensing module 2022. In this embodiment, adhesive layer 2024 not only supports and attaches sensing module 2022 to the body of the patient, but it may also support and attach leads 2026 and the electrodes as well. The configuration shown in FIG. 20C may be particularly suitable for attachment to a chest of a patient over or near the heart thereof.

FIG. 20D depicts a patient-wearable device 2030 that comprises a first sensing module 2032 and a second sensing module 2034, each of which is attached to an adhesive layer 2036. Adhesive layer 2036 may be wrapped, for example, around an ankle of a patient, such that each of first sensing module 2032 and second sensing module 2034 are on opposing sides of a dorsalis pedis artery of the patient, or around a lower leg of a patient, such that each of first sensing module 2032 and second sensing module 2034 are on opposing sides of a posterior tibial artery of the patient. In an alternate embodiment, each of first sensing module 2032 and second sensing module 2034 may have its own adhesive layer to facilitate such attachment.

In certain embodiments, first sensing module 2032 may comprise an RF transmitter (or RF transceiver) and second sensing module 2034 may comprise an RF receiver (or RF transceiver). First sensing module 2032 may be attached to one side of an arm or leg of a patient and second sensing module 2034 may be attached to an opposite side of the arm or leg of the patient.

Once in position, first sensing module 2032 may transmit RF signals to second sensing module 2034 over a period of time. Variations in the RF signals that are received by second sensing module 2034 from first sensing module 2023 may be analyzed to determine pulsatile blood flow in the portion of the arm or the leg between first sensing module 2032 and second sensing module 2034.

In some embodiments, multiple patient-wearable devices may be used to monitor for and detect a vascular occlusion (e.g., a blood clot) in a patient. For example, multiple patient-wearable devices may be used to monitor for and detect a vascular occlusion in a patient who has recently undergone a vascular medical procedure such as but not limited to angioplasty and stenting, atherectomy, arteriovenous (AV) fistula, AV graft, thrombectomy, vascular bypass surgery, or open carotid or femoral endarterectomy.

Such monitoring/detection of vascular occlusions may be achieved for example, by attaching multiple patient-wearable devices (e.g., multiple instances of patient-wearable device 100 or any of the other patient-wearable devices described herein) to different locations on a body of a patient (e.g., after the patient has undergone a vascular medical procedure) and monitoring pulse strength indications periodically generated by the devices to detect a differential decrease in pulse strength at one of the body locations. For example, a patient-wearable device may be attached to one or both sides of the neck of the patient (over one or both carotid arteries), to each wrist of the patient, and to each ankle of the patient. A processing unit (e.g., separate from the patient-wearable devices but communicatively connected thereto, or integrated into one or more of the patient-wearable devices) may receive via wired or wireless communication links pulse strength indications or measurements from each of the patient-wearable devices. The pulse strength indications may be received or collected on a periodic basis (e.g., every few seconds). If a pulse strength indication from a particular patient-wearable device is observed to drop by more than a predetermined threshold below a baseline, then an alert may be generated as this may indicate the presence of a vascular occlusion in the patient. The baseline may be established for example, based on previous readings obtained from the same patient-wearable device and/or the other patient-wearable devices. By way of example, if a pulse strength indication or measurement generated by a patient-wearable device attached to the right wrist of the patient shows a sudden drop below the baseline by more than a predetermined threshold, this may indicate that there is a blood clot in the right arm of the patient. In such a case, the processing unit may generate one or more alerts. The alerts may comprise, for example and without limitation, one or more audible alerts, one or more visible alerts, one or more haptic alerts, and/or one or more electronic communications such as a notification that is sent to a monitoring device of a caregiver.

In certain embodiments, once a potential vascular occlusion has been identified in a body part of the patient (e.g., in the neck or in a limb of the patient) using the above-described method, a patient-wearable device such as that described above in reference to FIG. 8 may be attached to the relevant body part of the patient to better identify the precise location of the occlusion.

In some embodiments, multiple patient-wearable devices may be used to measure blood flow in an extremity of a patient relative to a baseline. Such a measurement may be useful, for example, in detecting peripheral artery disease (PAD) in a limb of a patient. For example, a first patient-wearable device may be attached to an upper arm of the patient (e.g., near the brachial artery) and a second patient-wearable device may be attached to an ankle of the patient and both devices may be used to generate one or more pulse rate indications or measurements. In further accordance with this example, the pulse strength indication(s)/measurement(s) generated on the upper arm may provide a baseline and the pulse strength indication(s)/measurement(s) generated on the ankle may be compared to this baseline. An index for the relevant leg may be generated, for example, by dividing a pulse strength indication for the ankle by the pulse strength indication of the upper arm. A relatively low index number may indicate narrowing or blockage of the arteries in the legs. Although this example involves attachment of the patient-wearable devices to the upper arm and ankle, this is not intended to be limiting. Different body locations may be used to provide the baseline pulse strength measurement and different body locations may be used to generate an extremity pulse strength measurement to compare to the baseline.

In some embodiments, a model trained via machine learning may be used to determine a pulse condition of a patient based on sensor data obtained by one or more patient-wearable devices or sensing modules. For example, a machine learning classifier may be trained and then used to determine whether, based on currently-captured sensor data, a particular component of a domain signal captured by a sensor comprises part of a pulse or subpulse or instead comprises non-relevant data. The data that is used to train the machine learning model may be obtained or derived from previously-captured sensor data associated with a patient (e.g., previously captured pulse, subpulse or ECG data). Furthermore, data obtained from sensors located on one part of a patient's body may be used to train a model that is then used to detect a pulse or subpulse on another party of the patient's body.

### III. Example Computer System Implementation

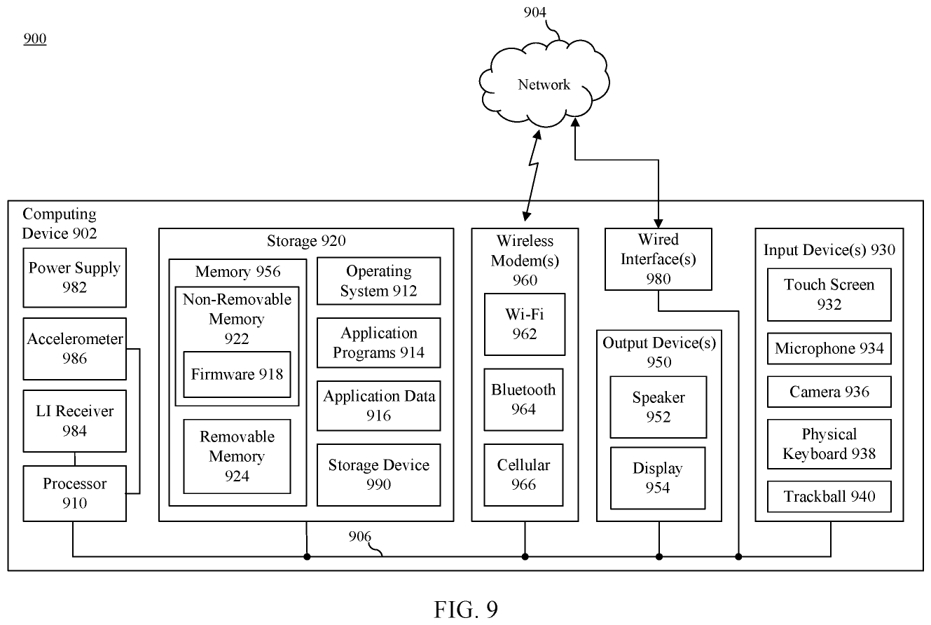

Examples of computing devices in which embodiments may be implemented are described as follows with respect to FIG. 9. FIG. 9 shows a block diagram of an exemplary computing environment 900 that includes a computing device 902. Computing device 902 is an example of computing device 628 of FIG. 6, which may include one or more of the components of computing device 902. In some embodiments, computing device 902 is communicatively coupled with devices (not shown in FIG. 9) external to computing environment 900 via network 904. Network 904 comprises one or more networks such as local area networks (LANs), wide area networks (WANs), enterprise networks, the Internet, etc., and may include one or more wired and/or wireless portions. Network 904 may additionally or alternatively include a cellular network for cellular communications.

Computing device 902 is described in detail as follows Computing device 902 can be any of a variety of types of computing devices. For example, computing device 902 may be a mobile computing device such as a handheld computer (e.g., a personal digital assistant (PDA)), a laptop computer, a tablet computer (such as an Apple iPad™), a hybrid device, a notebook computer (e.g., a Google Chromebook™ by Google LLC), a netbook, a mobile phone (e.g., a cell phone, a smart phone such as an Apple® iPhone® by Apple Inc., a phone implementing the Google® Android™ operating system, etc.), a wearable computing device (e.g., a head-mounted augmented reality and/or virtual reality device including smart glasses such as Google® Glass™, Oculus Rift® of Facebook Technologies, LLC, etc.), or other type of mobile computing device. Computing device 902 may alternatively be a stationary computing device such as a desktop computer, a personal computer (PC), a stationary server device, a minicomputer, a mainframe, a supercomputer, etc.

As shown in FIG. 9, computing device 902 includes a variety of hardware and software components, including a processor 910, a storage 920, one or more input devices 930, one or more output devices 950, one or more wireless modems 960, one or more wired interfaces 980, a power supply 982, a location information (LI) receiver 984, and an accelerometer 986. Storage 920 includes memory 956, which includes non-removable memory 922 and removable memory 924, and a storage device 990. Storage 920 also stores an operating system 912, application programs 914, and application data 916. Wireless modem(s) 960 include a Wi-Fi modem 962, a Bluetooth modem 964, and a cellular modem 966. Output device(s) 950 includes a speaker 952 and a display 954. Input device(s) 930 includes a touch screen 932, a microphone 934, a camera 936, a physical keyboard 938, and a trackball 940. Not all components of computing device 902 shown in FIG. 9 are present in all embodiments, additional components not shown may be present, and any combination of the components may be present in a particular embodiment. These components of computing device 902 are described as follows.

A single processor 910 (e.g., central processing unit (CPU), microcontroller, a microprocessor, signal processor, ASIC (application specific integrated circuit), and/or other physical hardware processor circuit) or multiple processors 910 may be present in computing device 902 for performing such tasks as program execution, signal coding, data processing, input/output processing, power control, and/or other functions. Processor 910 may be a single-core or multi-core processor, and each processor core may be single-threaded or multithreaded (to provide multiple threads of execution concurrently). Processor 910 is configured to execute program code stored in a computer readable medium, such as program code of operating system 912 and application programs 914 stored in storage 920. Operating system 912 controls the allocation and usage of the components of computing device 902 and provides support for one or more application programs 914 (also referred to as “applications” or “apps”). Application programs 914 may include common computing applications (e.g., e-mail applications, calendars, contact managers, web browsers, messaging applications), further computing applications (e.g., word processing applications, mapping applications, media player applications, productivity suite applications), one or more machine learning (ML) models, as well as applications related to the embodiments disclosed elsewhere herein.

Any component in computing device 902 can communicate with any other component according to function, although not all connections are shown for ease of illustration. For instance, as shown in FIG. 9, bus 906 is a multiple signal line communication medium (e.g., conductive traces in silicon, metal traces along a motherboard, wires, etc.) that may be present to communicatively couple processor 910 to various other components of computing device 902, although in other embodiments, an alternative bus, further buses, and/or one or more individual signal lines may be present to communicatively couple components. Bus 906 represents one or more of any of several types of bus structures, including a memory bus or memory controller, a peripheral bus, an accelerated graphics port, and a processor or local bus using any of a variety of bus architectures.

Storage 920 is physical storage that includes one or both of memory 956 and storage device 990, which store operating system 912, application programs 914, and application data 916 according to any distribution. Non-removable memory 922 includes one or more of RAM (random access memory), ROM (read only memory), flash memory, a solid-state drive (SSD), a hard disk drive (e.g., a disk drive for reading from and writing to a hard disk), and/or other physical memory device type. Non-removable memory 922 may include main memory and may be separate from or fabricated in a same integrated circuit as processor 910. As shown in FIG. 9, non-removable memory 922 stores firmware 918, which may be present to provide low-level control of hardware. Examples of firmware 918 include BIOS (Basic Input/Output System, such as on personal computers) and boot firmware (e.g., on smart phones). Removable memory 924 may be inserted into a receptacle of or otherwise coupled to computing device 902 and can be removed by a user from computing device 902. Removable memory 924 can include any suitable removable memory device type, including an SD (Secure Digital) card, a Subscriber Identity Module (SIM) card, which is well known in GSM (Global System for Mobile Communications) communication systems, and/or other removable physical memory device type. One or more of storage device 990 may be present that are internal and/or external to a housing of computing device 902 and may or may not be removable. Examples of storage device 990 include a hard disk drive, a SSD, a thumb drive (e.g., a USB (Universal Serial Bus) flash drive), or other physical storage device.

One or more programs may be stored in storage 920. Such programs include operating system 912, one or more application programs 914, and other program modules and program data. Examples of such application programs may include, for example, computer program logic (e.g., computer program code/instructions) for implementing any of the functions ascribed herein to computing device 628, as well as any of steps 506, 508 or 510 of flowchart 500 as previously described herein.

Storage 920 also stores data used and/or generated by operating system 912 and application programs 914 as application data 916. Examples of application data 916 include web pages, text, images, tables, sound files, video data, and other data, which may also be sent to and/or received from one or more network servers or other devices via one or more wired or wireless networks. Storage 920 can be used to store further data including a subscriber identifier, such as an International Mobile Subscriber Identity (IMSI), and an equipment identifier, such as an International Mobile Equipment Identifier (IMEI). Such identifiers can be transmitted to a network server to identify users and equipment.

A user may enter commands and information into computing device 902 through one or more input devices 930 and may receive information from computing device 902 through one or more output devices 950. Input device(s) 930 may include one or more of touch screen 932, microphone 934, camera 936, physical keyboard 938 and/or trackball 940 and output device(s) 950 may include one or more of speaker 952 and display 954. Each of input device(s) 930 and output device(s) 950 may be integral to computing device 902 (e.g., built into a housing of computing device 902) or external to computing device 902 (e.g., communicatively coupled wired or wirelessly to computing device 902 via wired interface(s) 980 and/or wireless modem(s) 960). Further input devices 930 (not shown) can include a Natural User Interface (NUI), a pointing device (computer mouse), a joystick, a video game controller, a scanner, a touch pad, a stylus pen, a voice recognition system to receive voice input, a gesture recognition system to receive gesture input, or the like. Other possible output devices (not shown) can include piezoelectric or other haptic output devices. Some devices can serve more than one input/output function. For instance, display 954 may display information, as well as operating as touch screen 932 by receiving user commands and/or other information (e.g., by touch, finger gestures, virtual keyboard, etc.) as a user interface. Any number of each type of input device(s) 930 and output device(s) 950 may be present, including multiple microphones 934, multiple cameras 936, multiple speakers 952, and/or multiple displays 954.

One or more wireless modems 960 can be coupled to antenna(s) (not shown) of computing device 902 and can support two-way communications between processor 910 and devices external to computing device 902 through network 904, as would be understood to persons skilled in the relevant art(s). Wireless modem 960 is shown generically and can include a cellular modem 966 for communicating with one or more cellular networks, such as a GSM network for data and voice communications within a single cellular network, between cellular networks, or between the mobile device and a public switched telephone network (PSTN). Wireless modem 960 may also or alternatively include other radio-based modem types, such as a Bluetooth modem 964 (also referred to as a “Bluetooth device”) and/or Wi-Fi 962 modem (also referred to as an “wireless adaptor”). Wi-Fi modem 962 is configured to communicate with an access point or other remote Wi-Fi-capable device according to one or more of the wireless network protocols based on the IEEE (Institute of Electrical and Electronics Engineers) 802.11 family of standards, commonly used for local area networking of devices and Internet access. Bluetooth modem 964 is configured to communicate with another Bluetooth-capable device according to the Bluetooth short-range wireless technology standard(s) such as IEEE 802.15.1 and/or managed by the Bluetooth Special Interest Group (SIG).

Computing device 902 can further include power supply 982, LI receiver 984, accelerometer 986, and/or one or more wired interfaces 980. Example wired interfaces 980 include a USB port, IEEE 1394 (FireWire) port, a RS-232 port, an HDMI (High-Definition Multimedia Interface) port (e.g., for connection to an external display), a DisplayPort port (e.g., for connection to an external display), an audio port, an Ethernet port, and/or an Apple® Lightning® port, the purposes and functions of each of which are well known to persons skilled in the relevant art(s). Wired interface(s) 980 of computing device 902 provide for wired connections between computing device 902 and network 904, or between computing device 902 and one or more devices/peripherals when such devices/peripherals are external to computing device 902 (e.g., a pointing device, display 954, speaker 952, camera 936, physical keyboard 938, etc.). Power supply 982 is configured to supply power to each of the components of computing device 902 and may receive power from a battery internal to computing device 902, and/or from a power cord plugged into a power port of computing device 902 (e.g., a USB port, an A/C power port). LI receiver 984 may be used for location determination of computing device 902 and may include a satellite navigation receiver such as a Global Positioning System (GPS) receiver or may include other type of location determiner configured to determine location of computing device 902 based on received information (e.g., using cell tower triangulation, etc.). Accelerometer 986 may be present to determine an orientation of computing device 902.

Note that the illustrated components of computing device 902 are not required or all-inclusive, and fewer or greater numbers of components may be present as would be recognized by one skilled in the art. For example, computing device 902 may also include one or more of a gyroscope, barometer, proximity sensor, ambient light sensor, digital compass, etc. Processor 910 and memory 956 may be co-located in a same semiconductor device package, such as being included together in an integrated circuit chip, FPGA, or system-on-chip (SOC), optionally along with further components of computing device 902.

In embodiments, computing device 902 is configured to implement any of the above-described features of flowcharts herein. Computer program logic for performing any of the operations, steps, and/or functions described herein may be stored in storage 920 and executed by processor 910.

As used herein, the terms “computer program medium,” “computer-readable medium,” and “computer-readable storage medium,” etc., are used to refer to physical hardware media. Examples of such physical hardware media include any hard disk, optical disk, SSD, other physical hardware media such as RAMs, ROMs, flash memory, digital video disks, zip disks, MEMs (microelectronic machine) memory, nanotechnology-based storage devices, and further types of physical/tangible hardware storage media of storage 920. Such computer-readable media and/or storage media are distinguished from and non-overlapping with communication media and propagating signals (do not include communication media and propagating signals). Communication media embodies computer-readable instructions, data structures, program modules or other data in a modulated data signal such as a carrier wave. The term “modulated data signal” means a signal that has one or more of its characteristics set or changed in such a manner as to encode information in the signal. By way of example, and not limitation, communication media includes wireless media such as acoustic, RF, infrared and other wireless media, as well as wired media. Embodiments are also directed to such communication media that are separate and non-overlapping with embodiments directed to computer-readable storage media.

As noted above, computer programs and modules (including application programs 914) may be stored in storage 920. Such computer programs may also be received via wired interface(s) 980 and/or wireless modem(s) 960 over network 904. Such computer programs, when executed or loaded by an application, enable computing device 902 to implement features of embodiments discussed herein. Accordingly, such computer programs represent controllers of the computing device 902.

Embodiments are also directed to computer program products comprising computer code or instructions stored on any computer-readable medium or computer-readable storage medium. Such computer program products include the physical storage of storage 920 as well as further physical storage types.

### IV. Conclusion

Various embodiments of a patient-wearable device for detecting a pulse condition of a patient and related systems, methods and computer program products have been described herein. As noted above, detecting a pulse condition of a patient may comprise detecting the presence or absence of a pulse of the patient, or the presence or absence of a subpulse of the patient. A subpulse should be understood to mean a spectrum of pulse that is less than reliability manually palpable. In an embodiment, detecting a subpulse may comprise detecting a pulse at a systolic blood pressure (SBP) of less than 80 mmHg. In a further embodiment, detecting a subpulse ay comprise detecting a pulse at an SBP of less than 60 mmHg. In a still further embodiment, detecting a subpulse may comprise detecting a pulse at an SBP of less than 52 mmHg.

While various embodiments of the present disclosure have been described above, it should be understood that they have been presented by way of example only, and not limitation. It will be understood by those skilled in the relevant art(s) that various changes in form and details may be made therein without departing from the spirit and scope of the invention as defined in the appended claims. Accordingly, the breadth and scope of the present invention should not be limited by any of the above-described exemplary embodiments, but should be defined only in accordance with the following claims and their equivalents.

What is claimed is:

1. A system, comprising:
a first patient-wearable device that is attachable to a first body location of a patient and configured to detect a pulse condition at the first body location, the first patient-wearable device comprising a first plurality of sensors;
a second patient-wearable device that is attachable to a second body location of the patient and configured to detect a pulse condition at the second body location, the second patient-wearable device comprising a second plurality of sensors; and
a processing unit that is communicatively connected to the first patient-wearable device and the second patient-wearable device and that is configured to selectively turn on and off individual ones of the sensors in the first plurality of sensors and the second plurality of sensors.

2. The system of claim 1, wherein at least one of the first patient-wearable device and the second patient-wearable device is configured to detect a subpulse condition at the respective body location.

3. The system of claim 1, wherein:
the first plurality of sensors and the second plurality include a same variety of sensor types.

4. The system of claim 3, wherein:
the first plurality of sensors includes at least a first inertial measurement unit (IMU) and a first acoustic sensor; and
the second plurality of sensors includes at least a second IMU and a second acoustic sensor.

5. The system of claim 4, wherein the first plurality of sensors and the second plurality of sensors further include, respectively:
a first echocardiogram (ECG) sensor and a second ECG sensor;
a first carbon dioxide sensor and a second carbon dioxide sensor; or
a first blood oxygen sensor and a second blood oxygen sensor.

6. The system of claim 1, wherein the processing unit comprises part of a computing device that is separate from and communicatively connected to the first patient-wearable device and the second patient-wearable device.

7. The system of claim 1, wherein the processing unit comprises part of the first patient-wearable device.

8. The system of claim 1, wherein the processing unit is configured to selectively turn on and off the individual ones of the sensors in the first plurality of sensors and the second plurality of sensors based on one or more of:
body locations to which the first patient-wearable device and the second patient-wearable device are respectively attached;
one or more factors associated with an environment of the patient; or
a type of medical procedure.

9. The system of claim 1, wherein the processing unit is configured to selectively turn on all sensors of a first type in the first plurality of sensors and turn off all sensors of the first type in the second plurality of sensors.

10. The system of claim 1, wherein the processing unit is configured to selectively turn on all sensors of a first type in the first plurality of sensors and the second plurality of sensors and to turn off all sensors of a second type in the first plurality of sensors and the second plurality of sensors.

11. A method performed by a processing unit, comprising:
establishing communication with a first patient-wearable device that is attached to a first body location of a patient and configured to detect a pulse condition at the first body location, the first patient-wearable device comprising a first plurality of sensors;
establishing communication with a second patient-wearable device that is attached to a second body location of the patient and configured to detect a pulse condition at the second body location, the second patient-wearable device comprising a second plurality of sensors; and
communicating control signals to the first patient-wearable device and the second patient-wearable device to selectively turn on and off individual ones of the sensors in the first plurality of sensors and the second plurality of sensors.

12. The method of claim 11, wherein at least one of the first patient-wearable device and the second patient-wearable device is configured to detect a subpulse condition at the respective body location.

13. The method of claim 11, wherein:
the first plurality of sensors and the second plurality include a same variety of sensor types.

14. The method of claim 13, wherein:
the first plurality of sensors includes at least a first inertial measurement unit (IMU) and a first acoustic sensor; and
the second plurality of sensors includes at least a second IMU and a second acoustic sensor.

15. The method of claim 14, wherein the first plurality of sensors and the second plurality of sensors further include, respectively:
a first echocardiogram (ECG) sensor and a second ECG sensor;
a first carbon dioxide sensor and a second carbon dioxide sensor; or
a first blood oxygen sensor and a second blood oxygen sensor.

16. The method of claim 11, wherein the processing unit comprises part of a computing device that is separate from and communicatively connected to the first patient-wearable device and the second patient-wearable device.

17. The method of claim 11, wherein the processing unit comprises part of the first patient-wearable device.

18. The method of claim 11, wherein selectively turning on and off the individual ones of the sensors in the first plurality of sensors and the second plurality of sensors comprises selectively turning on and off the individual ones of the sensors in the first plurality of sensors and the second plurality of sensors based on one or more of:
body locations to which the first patient-wearable device and the second patient-wearable device are respectively attached;
one or more factors associated with an environment of the patient; or
a type of medical procedure.

19. The method of claim 11, wherein selectively turning on and off the individual ones of the sensors in the first plurality of sensors and the second plurality of sensors comprises:
selectively turning on all sensors of a first type in the first plurality of sensors and turning off all sensors of the first type in the second plurality of sensors.

20. The method of claim 11, wherein selectively turning on and off the individual ones of the sensors in the first plurality of sensors and the second plurality of sensors comprises:
selectively turning on all sensors of a first type in the first plurality of sensors and the second plurality of sensors and turning off all sensors of a second type in the first plurality of sensors and the second plurality of sensors.

21. A computer-readable medium having instructions stored thereon that, when executed by a processing unit, cause the processing unit to perform operations comprising:
establishing communication with a first patient-wearable device that is attached to a first body location of a patient and configured to detect a pulse condition at the first body location, the first patient-wearable device comprising a first plurality of sensors;
establishing communication with a second patient-wearable device that is attached to a second body location of the patient and configured to detect a pulse condition at the second body location, the second patient-wearable device comprising a second plurality of sensors; and
communicating control signals to the first patient-wearable device and the second patient-wearable device to selectively turn on and off individual ones of the sensors in the first plurality of sensors and the second plurality of sensors.

22. The computer-readable medium of claim 21, wherein at least one of the first patient-wearable device and the second patient-wearable device is configured to detect a subpulse condition at the respective body location.

23. The computer-readable medium of claim 21, wherein:
the first plurality of sensors and the second plurality include a same variety of sensor types.

24. The computer-readable medium of claim 23, wherein:
the first plurality of sensors includes at least a first inertial measurement unit (IMU) and a first acoustic sensor; and
the second plurality of sensors includes at least a second IMU and a second acoustic sensor.

25. The computer-readable medium of claim 24, wherein the first plurality of sensors and the second plurality of sensors further include, respectively:
a first echocardiogram (ECG) sensor and a second ECG sensor;
a first carbon dioxide sensor and a second carbon dioxide sensor; or
a first blood oxygen sensor and a second blood oxygen sensor.

26. The computer-readable medium of claim 21, wherein the processing unit comprises part of a computing device that is separate from and communicatively connected to the first patient-wearable device and the second patient-wearable device.

27. The computer-readable medium of claim 21, wherein the processing unit comprises part of the first patient-wearable device.

28. The computer-readable medium of claim 21, wherein selectively turning on and off the individual ones of the sensors in the first plurality of sensors and the second plurality of sensors comprises selectively turning on and off the individual ones of the sensors in the first plurality of sensors and the second plurality of sensors based on one or more of:
body locations to which the first patient-wearable device and the second patient-wearable device are respectively attached;
one or more factors associated with an environment of the patient; or
a type of medical procedure.

29. The computer-readable medium of claim 21, wherein selectively turning on and off the individual ones of the sensors in the first plurality of sensors and the second plurality of sensors comprises:
selectively turning on all sensors of a first type in the first plurality of sensors and turning off all sensors of the first type in the second plurality of sensors.

30. The computer-readable medium of claim 21, wherein selectively turning on and off the individual ones of the sensors in the first plurality of sensors and the second plurality of sensors comprises:
selectively turning on all sensors of a first type in the first plurality of sensors and the second plurality of sensors and turning off all sensors of a second type in the first plurality of sensors and the second plurality of sensors.
